(19) United States
(12) Patent Application Publication
(10) Pub. No.: US 2010/0076089 A1
(43) Pub. Date: Mar. 25, 2010

(54) ANTIMICROBIAL GEL FORMULATIONS

(75) Inventors: Lu Wang, Pittsburg, CA (US); Azar Najafi, Danville, CA (US); Bahram Memarzadeh, San Carlos, CA (US); Kuldeepak Sharma, Fremont, CA (US); Kim Phuong Ho, Oakland, CA (US)

Correspondence Address: Hamilton DeSanctis & Cha, LLP, 3239 El Camino Real, Suite 220, Palo Alto, CA, 94306, US

(73) Assignee: NovaBay Pharmaceuticals, Inc.

(21) Appl. No.: 12/540,251

(22) Filed: Aug. 12, 2009

Related U.S. Application Data
(60) Provisional application No. 61/088,302, filed on Aug. 12, 2008.
(60) Provisional application No. 61/229,624, filed on Jul. 29, 2009.

(51) Int. Cl. A61K 31/131; A61P 31/00

(52) U.S. Cl. 514/665

(57) ABSTRACT

Disclosed herein are formulations comprising an N-halogenated or N,N-dihalogenated amine compound dispersed in a water-swellable polymer, wherein the compound is 90% stable for at least 30 days at about 25° C. Also disclosed are methods of treating or preventing infections caused by a bacterial, a microbial, a sporal, a fungal or a viral activity using such formulations.

### RELATED APPLICATIONS

This application claims the benefit of U.S. Provisional Application No. 61/088,302, filed on Aug. 12, 2008, and U.S. Provisional Application No. 61/229,624, filed on Jul. 29, 2009. The contents of both U.S. Application Nos. 61/088,302 and 61/229,624 are incorporated herein by reference in their entirety.

### FIELD

This invention relates to stable antimicrobial formulations of one or more N-halogenated or N,N-dihalogenated amine compounds dispersed in one or more water-swellable polymers.

### BACKGROUND

Certain chlorinated amine compounds, such as chlorinated taurine derivatives, have high antimicrobial activity and low cytotoxicity, and have been shown to be effective in killing bacteria, virus, fungi and other infectious agents. See, for example, U.S. Pat. No. 7,462,361 (M. Bassiri et al.). These compounds may be difficult to formulate for use in various applications, however, due to their reactivity.

While it may be desirable to formulate such compounds in polymers (e.g. gels) to impart properties such as adhesion to skin or mucous membranes, and sufficient residency times on such tissue, some compounds are not stable in, and react with or degrade in, the presence of certain formulation agents.

### SUMMARY

This disclosure describes stable antimicrobial formulations comprising an N-halogenated or N,N-dihalogenated amine compound dispersed in a water-swellable polymer wherein the compound is 90% stable for at least 30 days at about 25° C.

In certain implementations, the N-halogenated or N,N-dihalogenated amine compound may be a compound of Formula (I)

A-C(R¹R²)R(CH₂)ₙC(R³R⁴)—Y—Z (I)

or a derivative thereof, wherein

A is hydrogen, HalNH— or Hal₂N—, wherein Hal is a halogen selected from the group consisting of chloro, bromo and iodo;

R¹ is hydrogen or an optionally substituted group selected from the group consisting of alkyl, cycloalkyl, heteroalkyl, haloalkyl, aryl, heteroaryl and heterocycloalkyl, and —COOH;

R² is hydrogen or an optionally substituted group selected from the group consisting of alkyl, cycloalkyl, heteroalkyl, haloalkyl, aryl, heteroaryl and heterocycloalkyl, or R′ and R² together with the carbon atom to which they attach form an optionally substituted cycloalkyl or heterocycloalkyl group;

R is a carbon-carbon single bond or a divalent cycloalkylene radical with three to six carbon atoms,

n is 0 or an integer from 1 to 13;

R³ and R⁴ are each independently selected from the group consisting of hydrogen, fluoro, —NH₂, —NHHal, NHal₂, and an optionally substituted group selected from the group consisting of alkyl, cycloalkyl, heteroalkyl, aryl, heteroaryl, and heterocycloalkyl groups;

Y is selected from a group consisting of a single bond, —O—, —CF₂—, —CHF—, —C(═O)—, —C(═O)O—, —OC(═O)—, —C(═O)NR^{a}—, —NR^{a}C(═O)—, P(═O)(OR^{b})O—, —OP(═O)(OR^{b})—, —P(═O)(OR^{b})NR^{c}—, —NR^{C}P(═O)(OR^{b})—, —S(═O)₂, —S(═O)₂O—, —OS(═O)₂—, —S(═O)₂NR^{d}—, —NR^{d}S(═O)₂—, or heteroarylene wherein R^{a}, R^{b}, R^{c} and R^{d} are each independently selected from the group consisting of hydrogen, and optionally substituted alkyl, aryl, cycloalkyl, heteroalkyl, heteroaryl and heterocycloalkyl; a divalent (C₁₋₁₈)alkylene group in which, optionally, one or two methylene groups are replaced with a mono- or di-substituted methylene group; and a divalent (C₁₋₁₈)heteroalkylene group wherein the divalent (C₁₋₁₈)heteroalkylene group is a divalent (C₁₋₁₈)alkylene group in which, optionally, one or two methylene groups are replaced with 1 or 2 —NR′—, —O—, —S—, —S(═O)—, >C═O, —C(═O)O—, —OC(═O)—, —C(═O)NH—, —NHC(═O)—, —C(═O)NR′—, —NR′C(═O)—, —S(═O)₂—, —S(═O)₂NR′—, —S(═O)₂NH—, —NR′S(═O)₂— or —NHS(═O)₂— group, wherein R′ is selected from the group consisting of hydrogen, Cl, Br, and optionally substituted alkyl, aryl, cycloalkyl, heteroalkyl, heteroaryl, heterocycloalkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)arylC(—O)— and (C₆₋₁₀)aryl(C₁₋₄)alkylC(═O)— wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, or heterocycloalkyl(C₁₋₄alkyl, the heterocycloalkyl group containing 2-10 carbon atoms and 1 to 4 heteroatoms selected from N, O or S;

Z is selected from the group consisting of hydrogen, —CO₂H, —CONH₂, —SO₃H, —SO₂NH₂, —P(═O)(OH)₂, —B(OH)₂, —[X(R⁵)(R⁶)R⁷]Q, —S(═O)₂NR^{c}R^{d}, —S(═O)₂NHC(═O)R^{e}, S(═O)₂OC(═O)NR^{c}R^{d}, —S(═O)₂NR^{c}C(═O)NR^{c}R^{d} and —S(═O)₂(N═)C(OH)NR^{c}R^{d} wherein R^{c} and R^{d} are each independently hydrogen or is independently selected from the group consisting of (C₁₋₅)alkyl, (C₃₋₆)cycloalkyl, (C₁₋₅)alkylNHC(═O)—, (C₁₋₅)alkylC(═O)—, (C₆₋₁₀)arylC(═O)—, (C₆₋₁₀)aryl(C₁₋₄)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S, and R^{e} is hydrogen or is selected from the group consisting of (C₁₋₅)alkyl, (C₃₋₆)cycloalkyl, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S; or a salt, an amine oxide thereof, or a derivative or a bioisostere or a prodrug thereof;

X is selected from the group consisting of N, P, and S;

Q is a counterion or is absent;

R⁵ and R⁶ are each independently selected from the group consisting of alkyl, aryl, cycloalkyl, heteroalkyl, heteroaryl and heterocycloalkyl, each of which may be optionally substituted; or R⁵ and R⁶ together with the X atom to which they are attached form heterocycloalkyl group, which may be optionally substituted; and

R⁷ is alkyl, aryl, cycloalkyl, heteroalkyl, heteroaryl or heterocycloalkyl, each of which may be optionally substituted, and may further be 0 when X is N, with the proviso that R⁷ is absent when X is S;

with the proviso that if R is a divalent cycloalkylene radical, n will not exceed the integer 11.

In implementations, the water-swellable polymer is a poly(ethylene oxide) or poly(acrylic acid).

In implementations, the formulations are 95% stable for at least 35 days at temperatures ranging from about 2° C. to about 40° C. In other implementations, the formulations are 92% stable for at least 70 days at temperatures ranging from about 2° C. to about 40° C. In yet other implementations, the formulations are 95% stable for at least 180 days at temperatures ranging form about 2° C. to about 25° C.

In implementations, the formulation has enhanced antimicrobial activity over a formulation of an N-halogenated or N,N-dihalogenated amine compound with no polymer.

This disclosure also describes stable formulations comprising an N-halogenated or N,N-dihalogenated amine compound and one or more perfume agents. This disclosure also describes stable formulations comprising an N-halogenated or N,N-dihalogenated amine compound dispersed in a water-swellable polymer, and a perfume agent. In implementations, the perfume agent is cineole or 3-octanone.

This disclosure also describes methods of use of such stable formulations, including a method of preventing or treating an infection caused by a bacterial, a microbial, a sporal, a fungal or a viral activity, the method comprising the administration of an effective amount of the formulation. In one method, an effective amount of an antimicrobial formulation described herein is administered to the skin, hair, nail, or mucous membrane of a subject. In implementations, the infection can be a bacterial infection, including a bacterial infection of the skin.

One advantage of the formulations described herein is their stability, increasing their utility in various applications.

The details of one or more implementations are set forth in the accompanying figures and the description below. Other features, objects, and advantages will be apparent from the description and drawings, and from the claims.

### DESCRIPTION OF DRAWINGS

FIG. 1 is a graph illustrating the stability of a 1% formulation of N,N-dichloro-2,2-dimethyltaurine (“NVC-422”) in 1% Noveon® AA-1 Polycarbophil after being stored for various times up to 35 days at both 2-8° C. and 40° C., as measured by the relative concentration of NVC-422 at the measurement day relative to day zero.

FIG. 2 is a graph illustrating the stability, as in FIG. 1, of 2% formulation of NVC-422 in 1% Noveon® AA-1 Polycarbophil.

FIG. 3 is a graph illustrating the stability, as in FIG. 1, of a 1% formulation of NVC-422 in 1% Noveon® AA-1 Polycarbophil after being stored for 188 days at 2-8° C., 25° C. and 40° C.

FIG. 4 is a graph illustrating the stability, as in FIG. 1, of a 0.3% formulation of NVC-422 in 1% Polyox® 205.

FIG. 5 is a graph illustrating the stability, as in FIG. 1, of a 2% formulation of NVC-422 in an aqueous formulation of 0.5% cineole.

FIG. 6 shows results of a radial diffusion assay showing the antimicrobial activity of a solution of 0.6% NVC-422 alone and in formulations of 0.75% AA-1 (plate at left) and 3% Polyox® 205 (plate at right); the activity of the polymer alone is shown for comparison (at the left of each plate). Samples are in duplicate.

FIG. 7 shows results of a radial diffusion assay as in FIG. 8, of 1% NVC-422 alone and in perfume formulations containing cineole.

FIG. 8 shows results of a radial diffusion assay as in FIG. 9, of 1% NVC-422 in perfume formulations of camphor and 3-octanone.

FIG. 9 is a graph illustrating time-kill results of an aqueous solution of 0.3% NVC-422 alone and in 1% Polyox® against S. aureus. The results of Polyox® without NVC-422 are shown for comparison.

FIG. 10 is a graph illustrating time-kill results of an aqueous solution of 0.6% NVC-422 alone and in 1% Polyox® against S. aureus. The results of Polyox® without NVC-422 are shown for comparison.

FIG. 11 shows minimum biofilm eliminating concentration (MBEC) assay of 0.6% NVC-422 in aqueous solution and in a formulation of 0.75% AA-1, as compared to polymer with no NVC-422; water is used as the control.

FIG. 12 shows results of and MBEC assay as in FIG. 8, but in a formulation of 3% Polyox® 205.

### DETAILED DESCRIPTION

As utilized in accordance with the present disclosure, the following terms, unless otherwise indicated, shall be understood to have the following meanings:

“Alkyl” refers to a saturated, branched, or straight-chain monovalent hydrocarbon radical derived by the removal of one hydrogen atom from a single carbon atom of a parent alkane. Alkyl groups include, but are not limited to, methyl; ethyl; propyls such as propan-1-yl, propan-2-yl (iso-propyl), cyclopropan-1-yl, etc.; butyls such as butan-1-yl, butan-2-yl (sec-butyl), 2-methyl-propan-1-yl (iso-butyl), 2-methyl-propan-2-yl (t-butyl), cyclobutan-1-yl; pentyls; hexyls; octyls; dodecyls; octadecyls; and the like. An alkyl group comprises from 1 to about 22 carbon atoms, e.g., from 1 to 22 carbon atoms, e.g. from 1 to 12 carbon atoms, or, e.g., from 1 to 6 carbon atoms. A divalent group, such as a divalent “alkyl” group, a divalent “aryl” group, etc., may be referred to as an “alkylene” group or an “alkylenyl” group, an “arylene” group or an “arylenyl” group, respectively.

“Alkylcycloalkyl” refers to an alkyl radical, as defined above, attached to a cycloalkyl radical, as defined herein. Alkylcycloalkyl groups include, but are not limited to, methyl cyclopentyl, methyl cyclobutyl, ethyl cyclohexyl, and the like. An alkylcycloalkyl group comprises from 4 to about 32 carbon atoms, i.e. the alkyl group can comprise from 1 to about 22 carbon atoms and the cycloalkyl group can comprise from 3 to about 10 carbon atoms.

“Active agent” refers to a pharmaceutically active compound, for example an antifungal, antibacterial or antiviral compound. Active agents include compounds of Formulae I, IA, IB, IC, ID, II, and III (including salts and derivatives thereof).

“Acyl” refers to a radical —C(═O)R, where R is hydrogen, alkyl, cycloalkyl, cycloheteroalkyl, aryl, arylalkyl, heteroalkyl, heteroaryl, or heteroarylalkyl as defined herein, each of which may be optionally substituted, as defined herein. Representative examples include, but are not limited to formyl, acetyl, cylcohexylcarbonyl, cyclohexylmethylcarbonyl, benzoyl, benzylcarbonyl and the like.

“Acylamino” (or alternatively “acylamido”) refers to a radical —NR′C(═O)R, where R′ and R are each independently hydrogen, alkyl, cycloalkyl, cycloheteroalkyl, aryl, arylalkyl, heteroalkyl, heteroaryl, or heteroarylalkyl, as defined herein, each of which may be optionally substituted, as defined herein. Representative examples include, but are not limited to, formylamino, acetylamino (i.e., acetamido), cyclohexylcarbonylamino, cyclohexylmethyl-carbonylamino, benzoylamino (i.e., benzamido), benzylcarbonylamino and the like.

“Acyloxy” refers to a radical —OC(═O)R, where R is hydrogen, alkyl, cycloalkyl, cycloheteroalkyl, aryl, arylalkyl, heteroalkyl, heteroaryl or heteroarylalkyl, as defined herein, each of which may be optionally substituted, as defined herein. Representative examples include, but are not limited to, acetyloxy (or acetoxy), butanoyloxy, benzoyloxy and the like.

“Alkoxy” refers to a radical —OR where R represents an alkyl or cycloalkyl group as defined herein, each of which may be optionally substituted, as defined herein. Representative examples include, but are not limited to, methoxy, ethoxy, propoxy, butoxy, cyclohexyloxy and the like.

“Alkoxycarbonyl” refers to a radical —C(═O)-alkoxy where alkoxy is as defined herein.

“Alkylsulfonyl” refers to a radical —S(═O)₂R where R is an alkyl or cycloalkyl group as defined herein, each of which may be optionally substituted, as defined herein. Representative examples include, but are not limited to, methylsulfonyl, ethylsulfonyl, propylsulfonyl, butylsulfonyl and the like.

“Aryl” refers to an aromatic hydrocarbon group which may be a single aromatic ring or multiple aromatic rings which are fused together, linked covalently, or linked to a common group such as a methylene or ethylene moiety. Aryl groups include, but are not limited to, groups derived from, acenaphthylene, anthracene, azulene, benzene, biphenyl, chrysene, cyclopentadiene, diphenylmethyl, fluoranthene, fluorene, indane, indene, naphthalene, pentalene, perylene, phenalene, phenanthrene, pyrene, triphenylene, and the like. An aryl group comprises from 6 to about 20 carbon atoms, e.g., from 6 to 20 carbon atoms, e.g. from 6 to 10 carbon atoms.

“Arylalkyl” refers to an alkyl group in which one of the hydrogen atoms bonded to a carbon atom is replaced with an aryl group. Arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl may be used. An arylalkyl group comprises from 7 to about 42 carbon atoms, e.g. the alkyl group can comprise from 1 to about 22 carbon atoms and the aryl group can comprise from 6 to about 20 carbon atoms.

“Carboxylate” refers to the group RCO₂—, where R can be hydrogen, alkyl, aryl, cycloalkyl, heteroalkyl, or heteroaryl as defined herein, each of which may be optionally substituted, as defined herein.

“Carbamoyl” refers to the radical —C(═O)N(R)₂ where each R group is independently hydrogen, alkyl, cycloalkyl or aryl as defined herein, which may be optionally substituted, as defined herein.

“Cycloalkyl” refers to a saturated cyclic alkyl radical. Typical cycloalkyl groups include, but are not limited to, groups derived from cyclopropane, cyclobutane, cyclopentane, cyclohexane, and the like. A cycloalkyl group comprises from 3 to about 10 carbon atoms, e.g. from 3 to 10 carbon atoms, or, e.g. from 3 to 6 carbon atoms.

“Electron-withdrawing group” refers to atoms or functional groups which are electronegative either through a resonance effect or an inductive effect. Examples of such atoms and functional groups include, but are not limited to CO₂R⁰, —NO₂, —SO₃R⁰, —PO₃R⁰R⁰⁰, cyano, halogen (F, Cl, Br, I), and haloalkyl, where R⁰ and R⁰⁰ are independently H, alkyl, aryl, cycloalkyl, heteroalkyl, heteroaryl, or cycloheteroalkyl group, as defined herein, each of which may be optionally substituted.

“Halide” refers to a halogen bearing a negative charge, including fluoride, chloride, bromide, and iodide.

“Halo” refers to a halogen, including fluoro, chloro, bromo and iodo.

“Heteroalkyl” refer to an alkyl radical in which one or more of the carbon atoms (and any associated hydrogen atoms) are each independently replaced with the same or different heteroatomic groups. Heteroatomic groups include, but are not limited to —NR⁰—, —O—, —S—, —PH—, —P(O)₂—, —S(O)—, —S(O)₂—, and the like, where R⁰ is defined above. Heteroalkyl groups include, but are not limited to, —O—CH₃, —CH₂—O—CH₃, —S—CH₃, —CH, —S—CH₃, —NR⁰—CH₃, —CH, —NR⁰⁰—CH₃, and the like, where R⁰ and R⁰⁰ are defined above. A heteroalkyl group can comprise from 1 to about 22 carbon and hetero atoms, e.g., from 1 to 22 carbon and heteroatoms, e.g. from 1 to 12 carbon and hetero atoms, e.g., from 1 to 6 carbon and hetero atoms.

“Heteroaryl” refers to an aryl group in which one or more of the carbon atoms (and any associated hydrogen atoms) are each independently replaced with the same or different heteroatomic groups. Typical heteroatomic groups include, but are not limited to, —N—, —O—, —S— and —NR⁰—, where R⁰ is defined above. Typical heteroaryl groups include, but are not limited to, groups derived from acridine, carbazole, carboline, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. A heteroaryl group comprises from 5 to about 20 atoms, e.g., from 5 to 20 atoms, e.g. from 5 to 10 atoms.

“Heterocycloalkyl” refers to unsaturated cycloalkyl radical in which one or more carbon atoms (and any associated hydrogen atoms) are independently replaced with the same or different heteroatom. Typical heteroatoms to replace the carbon atom(s) include, but are not limited to, N, P, O, S, etc. Typical heterocycloalkyl groups include, but are not limited to, groups derived from epoxides, imidazolidine, morpholine, piperazine, piperidine, pyrazolidine, pyrrolidine, quinuclidine, and the like. The heterocycloalkyl group comprises between 3 and 10 carbon atoms.

“Hydroxy” refers to the group OH.

“Lower” refers to residues, e.g. alkyl residues, containing from 1 to 6 carbon atoms.

“Phosphate” refers to the group (R)ₙPO₄⁽³⁻ⁿ⁾⁻, where n is 0, 1 or 2 and R can be hydrogen, alkyl, aryl, cycloalkyl, heteroalkyl or heteroaryl as defined herein, each of which may be optionally substituted.

“Pharmaceutically acceptable” refers to that which is useful in preparing a pharmaceutical composition that is generally safe, non-toxic, and neither biologically nor otherwise undesirable, and includes that which is acceptable for veterinary as well as human pharmaceutical use.

“Prevent”, “preventing” and “prevention” of an infection refer to reducing the risk of a patient from developing an infection, or reducing the frequency or severity of an infection in a patient.

“Salt” refers to a cation or anion (e.g. a cationic or anionic compound of Formulae I, IA, IB, IC, ID, II, and III) coupled with an anion or a cation, either in solution or as a solid. Salts include pharmaceutically acceptable salts as well as solvent addition forms (solvates) of the same salt. Unless specified in reaction schemes, where certain compounds described herein are named or depicted as a particular salt (e.g. the chloride), all other salt forms are within the scope of this disclosure. Examples of salts suitable with the compositions and formulations described herein are described below.

“Stable” or “stability” refers to the ability of a given formulation to retain a minimum concentration of N-halogenated or N,N-dihalogenated amine compound at a certain temperature or temperature range over a certain amount of time. For example, a certain formulation may have a stability of 90% for at least 90 days when stored at about 25° C., meaning that it retains at least about 90% of the initial concentration of N-halogenated or N,N-dihalogenated amine compound under those conditions.

“Sulfate” refers to the group —OSO₃H or SO₄²⁻

“Sulfonate” refers to the group —OSO₂R, where R can be alkyl, aryl, cycloalkyl, heteroalkyl or heteroaryl.

“Subject” refers to any animal, including mammals such as humans.

A “substituted” group refers to a group wherein one or more (e.g. from 1 to 5, e.g. from 1 to 3) hydrogens are replaced with a substituent such as an acyl, alkoxy, alkyl, alkoxycarbonyl, alkylsulfonyl” amino, acyloxy, aryl, carboxyl, carbamoyl, cycloalkyl, halo, heteroalkyl, heteroaryl, cycloheteroaryl, oxo, hydroxy, acylamino, electron-withdrawing group, or a combination thereof. In certain aspects, substituents include, without limitation, cyano, hydroxy, nitro, fluoro, trifluoromethyl, methoxy, phenyl and carboxyl.

Aspects of the current disclosure relate to active agents comprising N-halogenated and N,N-dihalogenated compounds of Formula (I):

A-C(R¹R²)R(CH₂)ₙC(R³R⁴)—Y—Z (I)

or a derivative thereof, wherein

A is hydrogen, HalNH— or Hal₂N—, wherein Hal is a halogen selected from the group consisting of chloro, bromo and iodo;

R¹ is hydrogen or an optionally substituted group selected from the group consisting of alkyl, cycloalkyl, heteroalkyl, haloalkyl, aryl, heteroaryl and heterocycloalkyl, and —COOH;

R² is hydrogen or an optionally substituted group selected from the group consisting of alkyl, cycloalkyl, heteroalkyl, haloalkyl, aryl, heteroaryl and heterocycloalkyl, or R′ and R² together with the carbon atom to which they attach form an optionally substituted cycloalkyl or heterocycloalkyl group;

R is a carbon-carbon single bond or a divalent cycloalkylene radical with three to six carbon atoms,

n is 0 or an integer from 1 to 13;

R³ and R⁴ are each independently selected from the group consisting of hydrogen, fluoro, —NH₂, —NHHal, NHal₂, and an optionally substituted group selected from the group consisting of alkyl, cycloalkyl, heteroalkyl, aryl, heteroaryl and heterocycloalkyl groups;

Y is selected from a group consisting of a single bond, —O—, —CF₂—, —CHF—, —C(═O)—, —C(═O)O—, —OC(═O)—, —C(═O)NR^{a}—, —NR^{a}C(═O)—, P(═O)(OR^{b})O—, —OP(═O)(OR^{b})—, —P(═O)(OR^{b})NR^{c}—, —NR^{C}P(═O)(OR^{b})—, —S(═O)₂, —S(═O)₂O—, —OS(═O)₂—, —S(═O)₂NR^{d}—, —NR^{d}S(═O)₂—, or heteroarylene wherein R^{a}, R^{b}, R^{c} and R^{d} are each independently selected from the group consisting of hydrogen, and optionally substituted alkyl, aryl, cycloalkyl, heteroalkyl, heteroaryl and heterocycloalkyl; a divalent (C₁₋₁₈)alkylene group in which, optionally, one or two methylene groups are replaced with a mono- or di-substituted methylene group; and a divalent (C₁₋₁₈)heteroalkylene group wherein the divalent (C₁₋₁₈)heteroalkylene group is a divalent (C₁₋₁₈)alkylene group in which, optionally, one or two methylene groups are replaced with 1 or 2-NR′, —O—, —S—, —S(═O)—, >C═O, —C(═O)O—, —OC(═O)—, —C(═O)NH—, —NHC(═O)—, —C(═O)NR′—, —NR′C(═O)—, —S(═O)₂—, —S(═O)₂NR′—, —S(═O)₂NH—, —NR′S(═O)₂— or —NHS(═O)₂— group, wherein R′ is selected from the group consisting of hydrogen, Cl, Br, and optionally substituted alkyl, aryl, cycloalkyl, heteroalkyl, heteroaryl, heterocycloalkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)arylC(—O)— and (C₆₋₁₀)aryl(C₁₋₄)alkylC(═O)— wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, or heterocycloalkyl(C₁₋₄alkyl, the heterocycloalkyl group containing 2-10 carbon atoms and 1 to 4 heteroatoms selected from N, O or S;

Z is selected from the group consisting of hydrogen, —CO₂H, —CONH₂, —SO₃H, —SO₂NH₂, —P(═O)(OH)₂, —B(OH)₂, —[X(R⁵)(R⁶)R⁷]Q, —S(═O)₂NR^{c}R^{d}, —S(═O)₂NHC(═O)R^{e}, S(═O)₂C(═O)NR^{c}R^{d}, —S(═O)₂NR^{c}C(═O)NR^{c}R^{d} and —S(═O)₂(N═)C(OH)NR^{c}R^{d} wherein R^{c} and R^{d} are each independently hydrogen or is independently selected from the group consisting of (C₁₋₅)alkyl, (C₃₋₆)cycloalkyl, (C₁₋₅)alkylNHC(═O)—, (C₁₋₅)alkylC(═O)—, (C₆₋₁₀)arylC(═O)—, (C₆₋₁₀)aryl(C₁₋₄)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S, and R^{e} is hydrogen or is selected from the group consisting of (C₁₋₅)alkyl, (C₃₋₆)cycloalkyl, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S; or a salt, an amine oxide thereof, or a derivative or a bioisostere or a prodrug thereof;

X is selected from the group consisting of N, P, and S;

Q is a counterion or is absent;

R⁵ and R⁶ are each independently selected from the group consisting of alkyl, aryl, cycloalkyl, heteroalkyl, heteroaryl and heterocycloalkyl, each of which may be optionally substituted; or R⁵ and R⁶ together with the X atom to which they are attached form heterocycloalkyl group, which may be optionally substituted; and

R⁷ is alkyl, aryl, cycloalkyl, heteroalkyl, heteroaryl or heterocycloalkyl, each of which may be optionally substituted, and may further be 0 when X is N, with the proviso that R⁷ is absent when X is S;

with the proviso that if R is a divalent cycloalkylene radical, n will not exceed the integer 11.

In one aspect, the amides as represented herein are —NRpRq amides of sulfonic acid, carboxylic acid and phosphoric acids, wherein Rp and Rq independently are selected from the group consisting of hydrogen, (C₁₋₆)alkyl and aryl.

In certain compounds of Formula (I), A is HalNH—. In other compounds of Formula (I), A is Hal₂N—.

In certain compounds of Formula (I), R² is an optionally substituted group selected from the group consisting of alkyl, cycloalkyl, heteroalkyl, haloalkyl, aryl, heteroaryl and heterocycloalkyl, or R¹ and R² together with the carbon atom to which they attach form an optionally substituted cycloalkyl or heterocycloalkyl group. For example, R¹ and R² together with the carbon atom to which they attach can form a cyclopentyl group.

In certain compounds of Formula (I), R¹ and R² are each independently optionally substituted alkyl. For example, R¹ and R² may both be methyl. As another example, R¹ can be methyl and R² can be ethyl. In yet another example, R¹ can be methyl and R² can be 2-methylpropyl.

In certain compounds of Formula (I), R is a carbon-carbon single bond. In certain compounds of Formula (I), n is an integer from 1 to 3.

In certain compounds of Formula (I), R³ and R⁴ are both hydrogen.

In certain compounds of Formula (I), Y is a single bond. In other compounds of Formula (I), Y is a divalent (C₁₋₁₈)heteroalkylene group wherein the divalent (C₁₋₁₈)heteroalkylene group is a divalent (C₁₋₁₈)alkylene group in which, optionally, one or two methylene groups are replaced with 1 or 2 —NR′—, —O—, —S—, —S(═O)—, >C═O, —C(═O)O—, —OC(═O)—, —C(═O)NH—, —NHC(═O)—, —C(═O)NR′—, —NR′C(═O)—, —S(O)₂—, —S(═O)₂NR′—, —S(═O)₂NH—, NR′S(═O)₂— or —NHS(═O)₂—, wherein R′ is selected from the group consisting of hydrogen, Cl, Br, and optionally substituted alkyl, aryl, cycloalkyl, heteroalkyl, heteroaryl, heterocycloalkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)arylC(═O)— and (C₆₋₁₀)aryl(C₁₋₄)alkylC(—O)— wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, or heterocycloalkyl(C₁₋₄) alkyl, the heterocycloalkyl group containing 2-10 carbon atoms and 1 to 4 heteroatoms selected from N, O or S. For example, Y can be —CH₂—S(═O)₂—(CH₂)₂—. In another example, Y can be —CH₂—C(═O)N(CH₃)—(CH₂)₂—.

In certain compounds of Formula (I), Z is —SO₃H. In other compounds of Formula (I), Z is —[X(R⁵)(R⁶)R⁷]Q wherein X is N, S, or P; R⁵, R⁶, and R⁷ are independently optionally substituted alkyl; and Q is a counterion. For example, Z can be —S(CH₃)₂⁺ and Q can be Cl⁻. In another example, Z can be —N(CH₃)₂(CH₂—CF₃)⁺ and Q can be CF.

In one variation of Formula (I), A is hydrogen or Hal₂N— wherein Hal is a halogen selected from the group consisting of chloro, bromo and iodo. In another variation, Z is hydrogen, —COOH, —CONH₂, —SO₃H, SO₂NH₂, —P(═O)(OH)₂ or —B(OH)₂. In another variation, R¹ is hydrogen, C₁₋₆alkyl or the group —COOH; and R² is hydrogen or C₁₋₆alkyl, or R¹ and R² together with the carbon atom to which they attach form a (C₃-C₆)cycloalkyl ring. In another variation, R³ is hydrogen, C₁₋₆alkyl or NH₂ or —NHal₂; and R⁴ is hydrogen or C₁₋₆alkyl. In one variation, in the divalent cycloalkylene radical or in the divalent radical —(CH₂)ₙ—, one hydrogen may be substituted with —NHal₂.

In one variation of Formula (I), A is hydrogen, Hal₂N— or HalHN, wherein Hal is halogen selected from the group consisting of chloro, bromo and iodo; R′ is hydrogen, (C₁₋₆)alkyl or the group —COOH; R² is hydrogen or (C₁₋₆)alkyl, or R¹ and R² together with the carbon atom to which they attach form a (C₃₋₆)cycloalkyl ring; R is a carbon-carbon single bond or a divalent cycloalkylene radical with three to six carbon atoms; n is 0 or an integer from 1 to 13; R³ is hydrogen, (C₁₋₆)alkyl, —NHHal, or —NHal₂; R⁴ is hydrogen or (C₁₋₆)alkyl; Y is a single bond; and Z is selected from the group consisting of hydrogen, —SO₃H, —SO₂NH₂, —P(═O)(OH)₂ and —B(OH)₂. Within this aspect, if R is a divalent cycloalkylene radical, n will not exceed the integer 11. In the divalent cycloalkylene radical or in the divalent radical —(CH₂)ₙ— one hydrogen may be substituted with —NHal₂.

Compounds of Formula (I) may contain up to a total of three —NHal₂ or NHHal groups, for example, one or two —NHal₂ or —NHHal groups. In certain aspects, compounds of Formula (I) contain one —NHal₂ group, which may be in the alpha-, beta-, gamma-, delta-, epsilon- or omega-position of an acidic R¹ (if R¹ is —COOH) or Z group.

Another aspect of the current disclosure relates to compounds of Formula (IA)

A-C(R¹R⁰)R(CH₂)ₙ—C(R³R⁴)—X′ (IA)

or a derivative thereof, wherein

A is hydrogen, HalNH— or Hal₂N— wherein Hal is halogen selected from the group consisting of chloro, bromo and iodo;

R¹ is hydrogen, C₁₋₆alkyl or the group —COOH;

R⁰ is hydrogen or C₁₋₆alkyl; or R¹ and R⁰ together with the carbon atom to which they attach form a (C₃-C₆)cycloalkyl ring;

R is a carbon-carbon single bond or a divalent cycloalkylene radical with three to six carbon atoms;

n is 0 or an integer from 1 to 13;

R³ is hydrogen, C₁₋₆alkyl, NH₂ or NHal₂;

R⁴ is hydrogen or C₁₋₆alkyl; and

X′ is hydrogen, —COOH, —CONH₂, —SO₃H, —SO₂NH₂, —P(═O)(OH)₂ or —B(OH)₂;

with the proviso that if R is a divalent cycloalkylene radical n will not exceed the integer 11.

In the divalent cycloalkylene radical or in the divalent radical —(CH₂)ₙ— one hydrogen may be substituted with —NHHal or —NHal₂.

Another aspect of the current disclosure relates to N-halogenated and N,N-dihalogenated compounds of Formula (IB)

A-C(R¹R²)—C(R³R⁴)—Y—Z (IB)

or derivative thereof, wherein

A is selected from the group consisting of hydrogen, Hal₂N—, and HalHN wherein Hal is halogen selected from the group consisting of chloro and bromo;

R¹ and R² are each independently selected from the group consisting of (C₁₋₅)alkyl, heteroalkyl, halo(C₁₋₅)alkyl, (C₃₋₆)cycloalkyl, (C₃₋₆)cycloalkyl(C₁₋₃)alkyl, (C₆₋₁₀)aryl(C₁₋₄)alkyl, (C₆₋₁₄)aryl, heteroaryl, and (C₃₋₄₀)heterocycloalkyl, or R¹ and R², together with the carbon atom to which they are attached to form a (C₃₋₁₂)cycloalkyl or (C₃₋₁₂)heterocycloalkyl;

R³ and R⁴ are each independently selected from the group consisting of hydrogen, fluoro, (C₁₋₅)alkyl, heteroalkyl, halo(C₁₋₅)alkyl, (C₃₋₆)cycloalkyl, (C₃₋₆)cycloalkyl(C₁₋₃)alkyl, (C₆₋₁₀)aryl(C₁₋₄)alkyl, (C₆₋₁₄)aryl, heteroaryl and (C₃₋₄₀)heterocycloalkyl, or R³ and R⁴ together with the carbon atom to which they are attached to form a (C₃₋₁₂)cycloalkyl or (C₃₋₁₂) heterocycloalkyl;

Y is selected from a group consisting of single bond, —O—, a divalent (C₁₋₁₈)alkylene group in which optionally one or two methylene groups are replaced with a mono- or di-substituted methylene group, and a (C₁₋₁₈)heteroalkylene group; and

Z is selected from the group consisting of —SO₃H, —SO₂NH₂ and —P(—O)(OH)₂;

with the proviso that when R¹ is (C₁₋₅)alkyl or when R¹ and R² together with the carbon atom to which they attach form a (C₃₋₆)cycloalkyl, then Y must be —O— or a divalent (C₁₋₁₈)alkylene group wherein one or two methylene groups are replaced with a substituted methylene group or Y must be a divalent (C₁₋₁₈)heteroalkylene group wherein the (C₁₋₁₈)heteroalkylene group is a (C₁₋₁₈)alkylene group where one or two methylene groups are replaced with a —NR′—, —O—, —S—, —S(═O)— or —S(═O)₂—, wherein R′ is hydrogen or is selected from the group consisting of Cl, Br, (C₁₋₅)alkyl, (C₃₋₆)cycloalkyl, (C₆₋₁₀)aryl(C₁₋₄)alkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)arylC(—O)—, (C₆₋₁₀)aryl(C₁₋₄)alkylC(═O)—, (C₆₋₁₄)aryl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S, and wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, or heterocycloalkyl(C₁₋₄)alkyl, the heterocycloalkyl group containing 2-10 carbon atoms and 1 to 4 heteroatoms selected from N, O or S.

Another aspect of the disclosure relates to compounds of Formula (IB) wherein R¹ and R² together with the carbon atoms to which they are attached form a ring system with 4 to 7 carbon ring members, wherein optionally one or two ring members are nitrogen.

Another aspect of the current disclosure relates to N-halogenated and N,N-dihalogenated compounds of Formula (IC)

A-C(R¹R²)(CH₂)ₙY(CH₂)ₘ—Z (IC)

or derivative thereof, wherein

A is HalHN— or Hal₂N—, wherein Hal is halogen selected from the group consisting of chloro and bromo;

R¹ and R² are each independently selected from the group consisting of (C₁₋₆)alkyl, aryl, cycloalkyl, heteroalkyl, heteroaryl and heterocycloalkyl, each of which may be optionally substituted; or R¹ and R² together with the carbon atom to which they are attached form a cycloalkyl or heterocycloalkyl group, each of which may be optionally substituted;

Y is selected from the group consisting of a single bond, —O—, —CF₂—, —CHF—, —C(═O)—, —C(═O)O—, —OC(═O)—, —C(═O)NR^{a}—, —NR^{a}C(═O)—, —P(═O)(OR^{b})O—, —OP(═O)(OR^{b})—, —P(═O)(OR^{b})NR^{c}—, —NR^{c}P(═O)(OR^{b})—, —S(═O)₂, —S(═O)₂O—, —OS(═O)₂—, —S(═O)₂NR^{d}—, —NR^{d}S(═O)₂— or heteroarylene, wherein R^{a}, R^{b}, R^{c} and R^{d} are each independently selected from the group consisting of hydrogen, and optionally substituted alkyl, aryl, cycloalkyl, heteroalkyl, heteroaryl and heterocycloalkyl;

Z is —[X(R⁵)(R⁶)R⁷]Q, wherein X, Q, R⁵, R⁶, and R⁷ are defined as in Formula (I) above;

n is 0 or is an integer from 1 to 12; and

m is an integer from 1 to 12.

Another aspect of the current disclosure relates to the following N-halogenated and N,N-dihalogenated compounds of Formula (ID)

A-C(R¹R²)(CH₂)ₙC(R³R⁴)—Z (ID)

or derivative thereof, wherein

A is hydrogen, HalNH— or Hal₂N—, wherein Hal is halogen selected from the group consisting of chloro, bromo and iodo;

R¹ and R² are each independently selected from an optionally substituted group selected from the group consisting of (C₁₋₆)alkyl, cycloalkyl, heteroalkyl, heteroaryl and heterocycloalkyl, or R¹ and R² together with the carbon atom to which they attach form a (C₃₋₆)cycloalkyl ring;

n is 0 or an integer from 1 to 13;

R³ and R⁴ are independently selected from the group consisting of hydrogen, fluoro, and an optionally substituted group selected from the group consisting of alkyl, cycloalkyl, heteroalkyl, heteroaryl, and heterocycloalkyl groups;

Z is selected from the group consisting of, —SO₃H, —SO₂NH₂, —P(═O)(OH)₂, —B(OH)₂ and —[X(R⁵)(R⁶)R⁷]Q, wherein X, Q, R⁵, R⁶ and R⁷ are defined as in Formula (I) above.

Another aspect of the current disclosure relates to an N-halogenated and N,N-dihalogenated compounds of Formula (II)

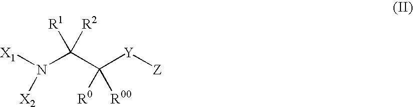

wherein:

X₁ is chloro or bromo;

X₂ is hydrogen or is selected from the group consisting of chloro, bromo, (C₁₋₅)alkyl, (C₃₋₆)cycloalkyl, (C₃₋₆)cycloalkyl, (C₁₋₃)alkyl and halo(C₁₋₅)alkyl;

R¹ and R² are each independently selected from the group consisting of (C₁₋₅)alkyl, halo(C₁₋₅)alkyl, (C₃₋₁₂)cycloalkyl, (C₃₋₆)cycloalkyl(C₁₋₃)alkyl, (C₆₋₁₀)aryl(C₁₋₄)alkyl, (C₆₋₁₄)aryl and heterocycloalkyl containing 2-10 carbon atoms and 1 to 4 heteroatoms selected from N, O or S, or R¹ and R² together with the carbon atom to which they are attached to form a (C₃₋₄₂)carbocyclic or (C₃₋₁₂)heterocyclyl with at least one heteroatom selected from N, O, or S in the ring;

R⁰ and R⁰⁰ are each independently hydrogen, fluoro or are the same as R¹ and R²; or

R¹ and R⁰ together with the carbon atoms to which they are attached form a ring with 4 to 7 carbon ring members, wherein optionally one or two ring members are nitrogen and optionally R⁰⁰ and R² together with the two carbon atoms to which they attach form a double bond; or

when X₁ is chloro or bromo, X₂ together with R⁰ form an alkylenyl group with 1 to 4 carbon atoms, the alkylenyl group together with —NX₁— and the carbon atom having the R¹ and R² groups and the carbon atom having the R⁰⁰ and the —Y—Z groups form a saturated heterocyclic ring in which one or two methylene groups may be replaced with a substituted methylene group, the substituents being fluoro, chloro or (C₁₋₅)alkyl, or one or two methylene groups may be replaced with —NR′— or >C═O, wherein R′ is as defined below;

Y is selected from the group consisting of single bond, —O—, and a divalent (C₁₋₁₈)alkylenyl group in which optionally one or two methylene groups are replaced with a mono- or di-substituted methylene group, or optionally where one or two methylene groups are replaced with 1 or 2 —NR′—, —O—, —S—, —S(═O)—, >C═O, —C(═O)O—, —OC(═O)—, —C(═O)NH—, —NHC(═O)—, —C(═O)NR′—, —NR′C(═O)—, —S(═O)₂—, —S(═O)₂NR′—, —S(═O)₂NH— or —NR′S(═O)₂— where R′ is hydrogen or is selected from the group consisting of Cl, Br, (C₁₋₅)alkyl, (C₃₋₆)cycloalkyl, (C₆₋₁₀)aryl, (C₆₋₁₀)aryl(C₁₋₄)alkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)aryl C(═O)—, (C₆₋₁₀)aryl(C₁₋₄)alkylC(═O)—, (C₆₋₁₄)aryl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S, wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀)aryl(C₁₋₄)alkyl or heteroaryl comprising 4 to 10 ring atoms with at least one heteroatom selected from O, S and N in the ring, or heterocyclyl(C₁₋₄)alkyl, the heterocycloalkyl group containing 2-10 carbon atoms and 1 to 4 heteroatoms selected from N, O or S;

wherein when R¹ is (C₁₋₅)alkyl or R¹ and R² together with the carbon atom to which they attach form a (C₃₋₆)cycloalkyl, then X₂ must be (C₁₋₅)alkyl, (C₃₋₆)cycloalkyl, (C₃₋₆)cycloalkyl-(C₁₋₃)alkyl, or halo(C₁₋₅)alkyl; or

when R¹ is (C₁₋₅)alkyl, then R² must be halo(C₁₋₅)alkyl, (C₃₋₁₂)cycloalkyl or (C₃₋₆)cycloalkyl-(C₁₋₃) alkyl; or

when R¹ is (C₁₋₅)alkyl or R¹ and R² together with the carbon atom to which they attach form a (C₃₋₆)cycloalkyl, then Y must be —O— or a divalent (C₁₋₁₈) alkylenyl group wherein one or two methylene groups are replaced with a substituted methylene group or by —NR′—, —O—, —S—, —S(═O)— or —S(═O)₂—, where R′ is hydrogen or is selected from the group consisting of Cl, Br, (C₁₋₅)alkyl, (C₃₋₆)cycloalkyl, (C₆₋₁₀)aryl, (C₆₋₁₀)aryl(C₁₋₄)alkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)arylC(═O)—, (C₆₋₁₀)aryl(C₁₋₄)alkylC(═O)—, (C₆₋₁₄)aryl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S, wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, or heterocyclyl(C₁₋₄alkyl, the heterocycloalkyl group containing 2-10 carbon atoms and 1 to 4 heteroatoms selected from N, O or S;

Z is selected from the group consisting of —SO₃H, —PO₃H₂, a salt or ester thereof, and an acid isostere thereof but not —C(═O)OH; or is selected from the group consisting of —S(═O)₂NR^{c}R^{d}, —S(═O)₂NHC(═O)R^{e}, S(═O)₂C(═O)NR^{c}R^{d}, —S(═O)₂NR^{c}C(═O)NR^{c}R^{d} and —S(═O)₂(N═)C(OH)NR^{c}R^{d}, wherein R^{c} and R^{d} are each independently hydrogen or is independently selected from the group consisting of (C₁₋₅)alkyl, (C₃₋₆)cycloalkyl, (C₁₋₅)alkylNHC(═O)—, (C₁₋₅)alkylC(═O)—, (C₆₋₁₀)arylC(═O)—, (C₆₋₁₀)aryl(C₁₋₄)C(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocyclyl containing 2-10 carbon atoms and 1 to 4 heteroatoms selected from N, O or S, and R^{e} is hydrogen or is selected from the group consisting of (C₁₋₅)alkyl, (C₃₋₆)cycloalkyl, (C₆₋₁₄)aryl,(C₆₋₁₀)aryl(C₁₋₄)alkyl, heteroaryl comprising 4 to 10 ring atoms with at least one heteroatom selected from O, S and N in the ring, and heterocyclyl containing 2-10 carbon atoms and 1 to 4 heteroatoms selected from N, O or S; or a salt, an amine oxide thereof, or a derivative or a bioisostere or a prodrug thereof.

Another aspect of the current disclosure relates to the following N-halogenated and N,N-dihalogenated compounds of Formula (III)

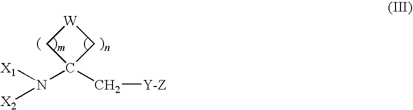

or a derivative therefor, wherein

X₁ is chloro or bromo;

X₂ is hydrogen or is selected from the group consisting of chloro, bromo, (C₁₋₅)alkyl, (C₃₋₆)cycloalkyl, (C₃₋₆)cycloalkyl(C₁₋₃)alkyl and halo(C₁₋₅)alkyl;

m and n are each independently an integer of 0, 1, 2, 3, 4 or 5, and m and n together is 2, 3, 4 or 5;

W is selected from the group consisting of —O—, —S—, —S(═O)—, —S(═O)₂—, —NR⁸—, —CR⁸R⁸—, >C═CR⁸R⁸, —N[C(═O)NHR⁹]—, —N[S(═O)₂R⁹]—, —N[S(═O)₂NHR⁹]—, —N[C(═O)R¹⁰]—, —NR⁹C(═O)—, >C═O and —N[C(═O)OR¹⁰]—;

Y is selected from a single bond, —O— and a divalent (C₁₋₁₈)alkylenyl group in which optionally one or two methylene groups are replaced with a mono- or di-substituted methylene group, or optionally where one or two methylene groups are replaced with 1 or 2 —NR′—,—O—, —S—, —S(═O)—, >C═O, —C(═O)O—, —OC(═O)—, —C(═O)NH—, —NHC(═O)—, —C(═O)NR′—, —NR′C(═O)—, —S(═O)₂—, —S(═O)₂NR′—, —S(═O)₂NH—, —NR′S(═O)₂— or —NHS(═O)₂—, where R′ is hydrogen or is selected from the group consisting of Cl, Br, (C₁₋₅)alkyl, (C₃₋₆)cycloalkyl, (C₆₋₁₀)aryl, (C₆₋₁₀)aryl(C₁₋₄)alkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)arylC(═O)—, (C₆₋₁₀)aryl(C₁₋₄alkylC(═O)—, (C₆₋₁₄)aryl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S, wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, or heterocyclyl(C₁₋₄alkyl, the heterocycloalkyl group containing 2-10 carbon atoms and 1 to 4 heteroatoms selected from N, O or S;

Z is selected from the group consisting of —SO₃H, —PO₃H₂, —S(═O)₂NR^{c}R^{d}, —S(═O)₂NHC(═O)R^{e}, —S(═O)₂NR^{c}C(═O)NR^{c}R^{d}, —S(═O)₂C(═O)NR^{c}R^{d} and —S(═O)₂(N═)C(OH)NR^{c}R^{d}, wherein R^{c} and R^{d} are each independently hydrogen or are selected from the group consisting of (C₁₋₅)alkyl, (C₁₋₅)alkylNHC(═O)—, (C₁₋₅)alkylC(═O)—, (C₃₋₆)cycloalkyl, aryl containing 6 to 14 ring atoms, (C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S, and R^{e} is hydrogen or is selected from the group consisting of (C₁₋₅)alkyl, (C₃₋₆)cycloalkyl, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocyclyl containing 2-10 carbon atoms and 1 to 4 heteroatoms selected from N, O or S;

R⁸ is each independently selected from the group consisting of hydrogen, (C₁₋₅)alkyl, (C₃₋₇)cycloalkyl, (C₃₋₆)cycloalkyl-(C₁₋₃)alkyl, (C₅₋₆)carbocyclyl where 1, 2 or 3 carbon ring members are optionally replaced by —NR′—, —O—, —S—, —S(═O)— or —S(═O)₂—, where R′ is hydrogen or is selected from the group consisting of Cl, Br, (C₁₋₅)alkyl, (C₃₋₆)cycloalkyl, (C₆₋₁₀)aryl, (C₆₋₁₀)aryl(C₁₋₄)alkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)arylC(═O)—, (C₆₋₁₀)aryl(C₁₋₄)alkylC(═O)—, (C₆₋₁₄)aryl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S, wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, or heterocyclyl(C₁₋₄alkyl, the heterocycloalkyl group containing 2-10 carbon atoms and 1 to 4 heteroatoms selected from N, O or S;

R⁹ is selected from the group consisting of (C₁₋₅)alkyl, (C₃₋₇)cycloalkyl, (C₃₋₆)cycloalkyl-(C₁₋₃)alkyl, (C₆₋₁₂)aryl and (C₅₋₆)carbocycloalkyl where 1, 2, or 3 carbon ring members are replaced by —NR′—, —O—, —S—, —S(═O)— or —S(═O)₂—, where R′ is hydrogen or is selected from the group consisting of (C₁₋₅)alkyl, (C₁₋₅)alkylNHC(═O)—, (C₁₋₅)alkylC(═O)—, (C₃₋₆)cycloalkyl, heterocycloalkyl containing 2-10 carbon atoms and 1 to 4 heteroatoms selected from N, O or S, (C₆₋₁₂)aryl, (C₆₋₁₀)aryl(C₁₋₄)alkyl, (C₆₋₁₂)heteroaryl, (C₁₋₆)alkylaryl, (C₆₋₁₂)aryl (C₁₋₆)alkyl, (C₁₋₅)alkylC(═O)—, (C₁₋₅)alkoxyC(═O)— and —S(═O)₂NH(C₁₋₅)alkyl;

R¹⁰ is selected from the group consisting of (C₁₋₅)alkyl, (C₁₋₅)alkoxy, (C₃₋₇)cycloalkyl, (C₃₋₆)cycloalkyl-(C₁₋₃)alkyl, (C₆₋₁₀)aryl, (C₆₋₁₀)aryloxy, (C₇₋₁₂)arylalkyl, (C₇₋₁₂)alkylaryl and (C₇₋₁₂)arylalkoxy.

In certain compounds of Formula (III), W is selected from the group consisting of —O—, —S(═O)₂—, —NR⁸—, —CR⁸R⁸—, >C═CR⁸R⁸, —N[C(═O)NHR⁹]—, —N[S(═O)₂R⁹]—, —N[S(═O)₂NHR⁹]—, —N[C(═O)R¹⁰]— and —N[C(═O)OR¹⁰]—; and R⁸, R⁹ and R¹⁰-are as defined above; Y is a single bond or a divalent (C₁₋₅)alkylenyl group; and Z is —SO₃H or —PO₃H₂; or a salt thereof.

Derivatives of the compounds described herein include pharmaceutically acceptable salts, C₁₋₆alkyl esters of carboxylates, sulfonates and phosphonates, and mono- and di-C₁₋₆alkyl amides of —NH₂ groups.

The above-described compositions include the following compounds (or a derivative thereof, as defined herein):
N,N-dichlorotaurine;
N,N-dichloro-2-methyltaurine;
N,N-dichloro-2,2,3,3-tetramethyl-β-alanine;
N,N-dichloro-2,2-dimethyltaurine;
N,N-dichloro-1,1,2,2-tetramethyltaurine;
N,N-dibromo-2,2-dimethyltaurine;
N,N-dibromo-1,1,2,2-tetramethyltaurine;
N,N-diiodotaurine;
N,N-dichloro-3,3-dimethylhomotaurine;
N,N-dichloro-2-methyl-2-aminoethanesulfonic acid;
N,N-dichloro-1-methyl-ethanesulfonic acid;
N,N-dichloro amino-trimethylene phosphonic acid;
N,N-dibromo-2-amino-5-phosphonopantanoic acid;
N,N-dichloroamino-ethylphosphonic acid dimethylester;
N,N-dichloroamino-ethylphosphonic acid diethylester;
N,N-dichloro-1-amino-1-methylethane phosphonic acid;
N,N-dichloro-1-amino-2-methylethane phosphonic acid;
N,N-dichloro-1-amino-2-methylpropane phosphonic acid;
N,N-dichloro-leucine phosphonic acid;
N,N-dichloro-4-amino-4-phosphonobutyric acid;
(±)N,N-dichloro-2-amino-5-phosphonovaleric acid;
N,N-dichloro-(+)-2-amino-5-phosphonovaleric acid;
N,N-dichloro d,l-2-amino-3-phosphonopropionic acid;
N,N-dichloro-2-amino-8-phosphonooctanoic acid;
N,N-dichloro-leucine boronic acid;
N,N-dichloro-β-alanine boronic acid;
N-chlorotaurine;
N-chloro-2-methyltaurine;
N-chloro-2,2,3,3-tetramethyl-β-alanine;
N-chloro-2,2-dimethyltaurine;
N-chloro-1,1,2,2-tetramethyltaurine;
N-bromo-2,2-dimethyltaurine;
N-bromo-1,1,2,2-tetramethyltaurine;
N-iodotaurine;
N-chloro-3,3-dimethylhomotaurine;
N-chloro-2-methyl-2-amino-ethanesulfonic acid; and
N-chloro-1-methyl-ethanesulfonic acid;
N-chloroamino-trimethylene phosphonic acid;
N-bromo-2-amino-5-phosphonopantanoic acid;
N-chloroamino-ethylphosphonic acid dimethylester;
N-chloroamino-ethylphosphonic acid diethylester;
N-chloro-1-amino-1-methylethane phosphonic acid;
N-chloro-1-amino-2-methylethane phosphonic acid;
N-chloro-1-amino-2-methylpropane phosphonic acid;
N-chloro-leucine phosphonic acid;
N-chloro-4-amino-4-phosphonobutyric acid;
(±)N-chloro-2-amino-5-phosphonovaleric acid;
N-chloro-(+)2-amino-5-phosphonovaleric acid;
N-chloro d,l-2-amino-3-phosphonopropionic acid;
N-chloro-2-amino-8-phosphonooctanoic acid;
N-chloro-leucine boronic acid;
N-chloro-β-alanine boronic acid;
(1-(dichloroamino)cyclohexylmethanesulfonic acid;
(1-(chloroamino)cyclohexyl)methanesulfonic acid;
2-(chloroamino)-N,N,N-2-tetramethylpropan-1-ammonium chloride;
2-(dichloroamino)-N,N,N-2-tetramethylpropan-1-ammonium chloride;
3-(chloroamino)-N,N,N-3-tetramethylbutan-1-ammonium chloride;
3-(dichloroamino)-N,N,N-3-tetramethylbutan-1-ammonium chloride;
1-(2-(dichloroamino)-2-methylpropyl)-1-methylpiperidinium chloride;
1-(2-(chloroamino)-2-methylpropyl)-1-methylpiperidinium chloride;
(2-(dichloroamino)-2-methylpropyl)dimethylsulfonium chloride;
(2-(chloroamino)-2-methylpropyl)dimethylsulfonium chloride;
(4-(dichloroamino)-4-methylpentyl)trimethylphosphonium chloride;
(4-(chloroamino)-4-methylpentyl)trimethylphosphonium chloride;
3-(3-(dichloroamino)-3-methylbutylsulfonyl)-N,N,N-trimethylpropan-1-aminium chloride;
3-(3-(chloroamino)-3-methylbutylsulfonyl)-N,N,N-trimethylpropan-1-aminium chloride;
2-(3-(dichloroamino)-3-methylbutylsulfonyl)-N,N,N-trimethylethanaminium chloride;

and
2-(3-(chloroamino)-3-methylbutylsulfonyl)-N,N,N-trimethylethanaminium chloride.

It will be appreciated that the common name “taurine” refers to “2-aminoethanesulfonic acid,” and that compounds referred to herein containing “taurine” contain this chemical motif. For instance, “N,N-dichlorotaurine” may also be referred to as “2-(dichloroamino)-ethanesulfonic acid” and “N,N-dichloro-2,2-dimethyltaurine” may also be referred to as “2-(dichloroamino)-2-methylpropanesulfonic acid.”

The N-halogenated or N,N-dihalogenated compounds described above may be neutral, cationic, or in a salt form. The compounds may be identified either by their chemical structure and/or chemical name. If the chemical structure and chemical name conflict, the chemical structure is determinative of the identity of the compound. The compounds may contain one or more chiral centers and/or double bonds and therefore, may exist as stereoisomers, such as double-bond isomers (i.e., geometric 20 isomers), enantiomers or diastereomers. Accordingly, when stereochemistry at chiral centers is not specified, the chemical structures depicted herein encompass all possible configurations at those chiral centers including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. Compounds may exist in unsolvated forms as well as solvated forms, including hydrated forms and as N-oxides.

Suitable salts include the following: (1) acid addition salts, formed with inorganic acids such as hydrochloric acid, hydrobromic acid, sulfuric acid, nitric acid, phosphoric acid, and the like; or formed with organic acids such as acetic acid, butyric acid, propionic acid, hexanoic acid, cyclopentanepropionic acid, glycolic acid, pyruvic acid, lactic acid, valeric acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like, made by conventional chemical means; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like, made by conventional chemical means.

Examples of acid addition salts include, but are not limited to, mineral or organic acid salts of basic residues such as substituted amides (for example, when —C(═O)NH— is present) or alkali or organic salts of acidic residues (for example, when —OP(═O)(OH) is present). Pharmaceutically acceptable salts include, but are not limited to, hydrohalides, sulfates, methosulfates (quaternary ammonium sulfates), methanesulfonates, toluenesulfonates, nitrates, phosphates, maleates, acetates, lactates, oxalates, fumerates, succinates, and the like. The pharmaceutically acceptable acid addition salts further include salts with hydrochloric, sulfonic, phosphoric, nitric acid, acetic, benzenesulfonic, toluenesulfonic, methanesulfonic acid, camphorsulfonic acid, oxalic acid, succinic acid, fumeric acid and other acids.

Lists of suitable salts are found, for example, in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa., (2005), at p. 732, Table 38-5, which are hereby incorporated herein by reference.

The N-halogenated or N,N-dihalogenated amine compounds described herein are in stable formulations comprising a water-swellable polymer, that is, a polymer that hydrates in water to form a viscous solution or suspension.

Examples of water-swellable polymers include poly(acrylic acid) polymers (e.g. Carbopol®, available from the Lubrizol Corporation) and poly (ethylene oxide) polymers (e.g. Polyox®, available from the Dow Chemical Company). Carbopol® homopolymers are polymers of acrylic acid crosslinked with allyl sucrose or allyl pentaerythritol. Carbopol® copolymers are polymers of acrylic acid and C₁₀-C₃₀ alkyl acrylate crosslinked with allyl pentaerythritol. Carbopol® interpolymers are a carbomer homopolymer or copolymer that contains a block copolymer of polyethylene glycol and a long chain alkyl acid ester. Suitable grades of carbopol include but not limited to Carbopol® homopolymers, Carbopol® copolymers, Carbopol® interpolymers, Noveon® AA-1 Polycarbophil (“AA-1”), Noveon® CA-1 Polycarbophil (calcium neutralized), Noveon® CA-2 Polycarbophil (calcium neutralized) and other commercially available grades of Carbopol® and Polycarbophil. Suitable grades of Polyox® include but not limited to: WSR N-10, WSR N-80, WSR N-750, WSR N-3000, WSR-205 (“Polyox® 205” of “Polyox” in certain figures), WSR-1105, WSR N-12K, WSR N-60K, WSR-301, WSR Coagulant, WSR-308 UCARFLOC Polymer 300, UCARFLOC Polymer 302, UCARFLOC Polymer 304 and UCARFLOC Polymer 309 available from Dow Chemical Company. Poly(1-vinyl-2-pyrrolidinone) (Povidone) may also be used.

The formulations may be made by mixing the N-halogenated or N,N-dihalogenated amine compound with the polymer using water. Water/oil emulsions, hydrating agents, wetting agents, surfactants, and the like may also be used. Concentrations of N-halogenated or N,N-dihalogenated amine compound may range from about 0.01% to about 5.0% (by weight). Concentrations of polymer may be from about 0.01% to about 10% (by weight) in water. For example, in certain implementations, the concentration of N-halogenated or N,N-dihalogenated compounds may range from about 0.1% to about 2.0% (by weight). Higher concentrations of polymer may be required to formulate higher concentrations of the N-halogenated or N,N-dihalogenated amine compound. For example, a 1% AA-1 formulation can hold up to about 2% N,N-dichloro-2,2-dimethyltaurine, whereas a 2% AA-1 formulation can hold up to about 3.5% of the same compound. These ratios may differ as different polymers and N-halogenated or N,N-dihalogenated amine compound are combined in a given formulation.

The formulations described herein were generally prepared as follows. Polymer was hydrated slowly in purified water with or without common pharmaceutical excipients such as sodium chloride, salts and buffers. A N-halogenated or N,N-dihalogenated amine compound (e.g. N,N-dichloro-2,2-dimethyltaurine, or “NVC-422”) was then added. The solution was then mixed and adjusted to pH between about 3.0 and about 9.0 using a suitable acid or base, e.g. NaOH and HCl.

Stable formulations described herein are at least 90% stable for at least 30 days at about 25° C. In certain implementations, stable formulations may have higher stability. The stability of a given formulation depends generally on the particular N-halogenated or N,N-dihalogenated amine compound and polymer used in the formulation. Stability, as described herein, is also generally a function of storage time and temperate.

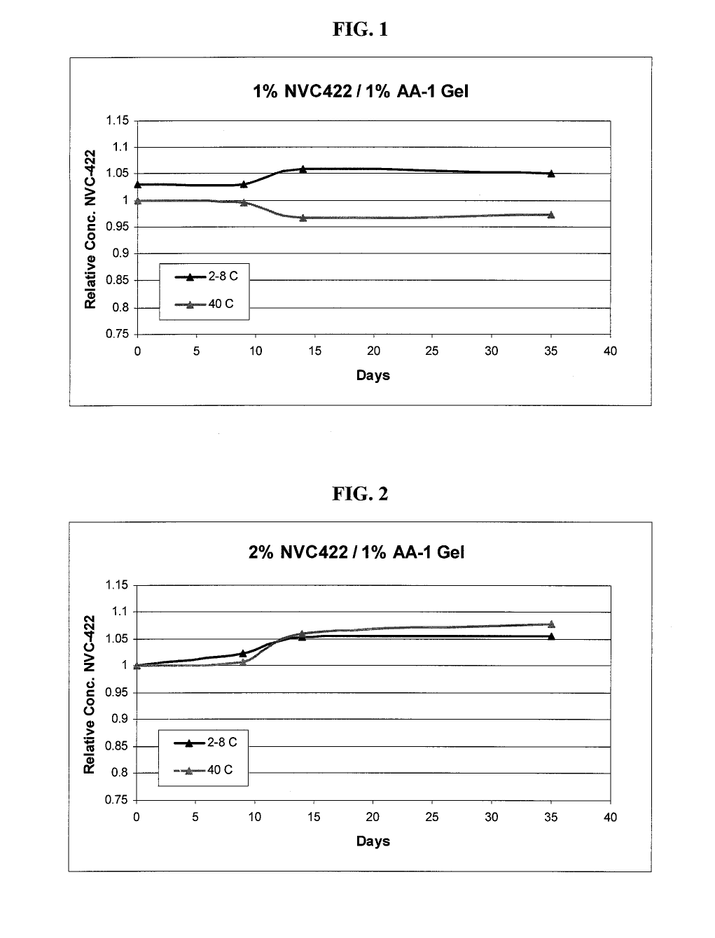

Referring to FIG. 1, a formulation of 1% N,N-dichloro-2,2-dimethyltaurine (NVC-422) in 1% AA-1 polymer retains at least 95% of the initial concentration of N,N-dichloro-2,2-dimethyltaurine for a period of at least 35 days when stored at a temperature of about 2° C. to about 8° C., and at about 40° C. (as measured by UV/Vis or HPLC). Thus, such a formulation is described as being 95% stable (or having 95% stability) for at least 35 days when stored from about 2° C. to about 40° C. Note that in this and other stability figures, the relative concentration of chlorinated amine compound may increase slightly over 100% due to, e.g., evaporation of water from the formulation.

Referring to FIG. 2, a formulation of 2% NVC-422 in 1% AA-1 has 95% stability for at least 35 days when stored from about 2° C. to about 40° C.

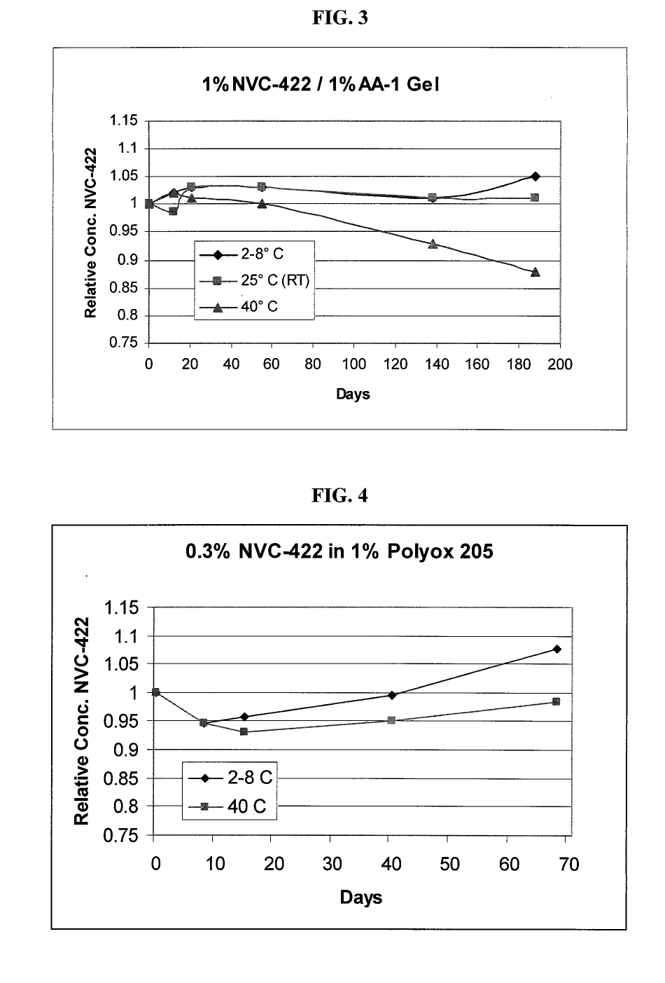

Referring to FIG. 3, a formulation of 1% NVC-422 in 1% AA-1 polymer has 95% stability for at least 188 days when stored at a temperature of about 2° C. to about 25° C., and has 85% stability for at least 188 days when stored at about 40° C.

Referring to FIG. 4, a formulation of 0.3% NVC-422 in 1% Polyox® 205 has 92% stability for at least 70 days when stored at about 2° C. to about 40° C.

Not all water-swellable polymers can be used to form stable formulations of the N-halogenated or N,N-dihalogenated amine compounds described herein. For example, it was observed that a formulation of 1% of NVC-422 in 2.5% Carbopol® Aqua-CC adjusted to pH 4.0 with HCl degraded to about 41% of the initial concentration of NVC-422 (as measured by UV/Vis or HPLC) between the time it took to prepare the sample and perform the stability analysis, and degraded to about 23% of the initial concentration by the second day at about 25° C. Thus, this formulation was deemed not stable. Furthermore, formulations of NVC-422 in Carbopol® R-1 NF and Carbopol® Ultrez 10 NF were also unstable. It was not possible to measure stabilities of those samples because the formulations immediately became cloudy or discolored after being prepared.

The stable formulations or compositions described herein may include one or more other constituents, including a solvent, co-solvent, gelling agent, humectant, film-forming agent, carrier, penetration enhancer, plasticizer, or other inactive ingredients, and combinations thereof.

Suitable solvents and co-solvents include water, alcohols (e.g. methanol, ethanol, propanols, etc.), and other solvents in which the N-halogenated and/or N,N-dihalogenated amine compounds and perfume agents are soluble.

The stable formulations may be altered with suitable acids and bases, for example with HCl and NaOH. In various implementations, the formulations may have a pH from about 3.0 to 9.0, e.g. from about 3.0 to about 7.0, e.g. about 3.0 to about 6.0, and e.g. from about 3.5 to about 4.5.

Stable formulations may include salts and buffers. For example, a saline solution (e.g. NaCl) may be used. Suitable buffers include, but are not limited to, Clark and Lubs solutions, pH 2.2-4.0 (Bower and Bates, J. Res Natn. Bur. Stand. 55, 197 (1955)); beta,beta-dimethylglutaric acid-NaOH buffer solutions, pH 3.2-7.0 (Stafford, Watson, and Rand, BBA 18, 318 (1955)); sodium acetate-acetic acid buffer solutions, pH 3.7-5.6; succinic acid-NaOH buffer solutions, pH 3.8-6.0 (Gomeri, Meth. Enzymol. 1, 141 (1955)); sodium cacodylate-HCl buffer solutions, pH 5.0-7.0 (Pumel, Bull. Soc. Chim. Biol. 30, 129 (1948)); Na₂HPO₄—NaH₂PO₄ buffer solutions, pH 5.8-7.0 (Gomeri and Sorensons, Meth. Enzmol. 1, 143 (1955)); potassium biphthalate/HCl, pH 3.0 to 3.8; potassium biphthalate/NaOH pH 4.0-6; KH₂PO₄/NaOH, pH 6.0-7.0; and monopotassium phosphate/NaOH, pH 6.0 to pH 8.0 or NaOH/boric acid, pH 7.8 to pH 8.0 (see OECD Guideline for Testing Chemicals “Hydrolysis as a Function of pH,” Adopted 12 May 1981, 111, pp. 10-11).

Stable formulations may also include pharmaceutically acceptable excipients which can be found in Remington: The Science and Practice of Pharmacy, R. Hendrickson, ed., 21st edition, Lippincott, Williams & Wilkins, Philadelphia, Pa., (2005) at pages 317-318, which is herein incorporated by reference in its entirety. Stable formulations may assume various forms, including suspensions, emulsions, ointments, creams, gels, lotions, pastes, and the like, as well as powders, mixtures of powders and the like, emulsions, suspensions as well as solutions and gaseous formulations, such as aerosols.

For certain applications, it may be desirable to impart a pleasant smell, or to mask an unpleasant small, of solutions comprising an N-halogenated or N,N-dihalogenated amine compound. Accordingly, in another aspect, a stable formulation may comprise one or more N-halogenated or N,N-dihalogenated amine compounds described herein and one or more perfume agents (i.e. fragrances, colognes, or perfumes). Any type of perfume agent compatible with the N-halogenated and N,N-dihalogenated amine compounds may be used. Such stable perfume agents will generally be in an aqueous solvent (e.g. water with or without acids, bases, buffers, etc.) but may also be formulated using other solvents, co-solvents, excipients, etc. described herein. Suitable perfume agents include alcohols, aldehydes, ketones, nitriles, and esters used in or known as perfumes and fragrances. Examples of suitable perfume agents include, without limitation, menthol, anethole, carvone, eugenol, limonene, ocimene, n-decylalcohol, citronellol, a-terpineol, methyl salicylate, methyl acetate, citronellyl acetate, cineole (e.g. 1,8-cineol, also known as eucalyptol), camphor, linalool, ethyl linalool, vanillin, thymol, isoamyl phenyl ether, isoborneol, isoborneol methyl ether, 2,2-dimethylbicyclo[2.2.1]heptane-3-carboxylic acid methyl ester, 2-tertiary pentyl cyclohexanyl acetate, 7-octen-2-ol-2,6-dimethyl acetate, 1-methyl-4-isopropyl cyclohexan-8-yl acetate, tetrahydrogeraniol, 2,6-dimethylheptan-2-ol, diphenyl methane, diphenyl oxide, alpha-fenchyl acetate, 1,3-dioxane-2,4,6-trimethyl-4-phenyl, 4-methyl-2-(2-methylpropyl)tetrahydro-2H-pyran-4-ol, ethyl tricyclo[5.2.1.0.2,6]decan-2-carboxylate, 2-methyldecanonitrile, 2-butyl-4,4,6-trimethyl-1,3-dioxane, 2-butyl-4,4,6-trimethyl-1,3-dioxane, limetol, 3,12-tridecadiene nitrile, methyl lavender ketone, octanal dimethyl acetal, orange flower ether (i.e. 4-(1-methoxy-1-methylethyl)-1-methylcyclohexene), p-tertiary butyl cyclohexanol, benzene pentanol, 3-octanol, 3,7-dimethyl-3-octanol, 2,6-dimethyl-2-octanol, 2-octanone, 3-octanone, thymyl methyl ether, ortho-tertiary butyl cyclohexanyl acetate, benzene, [2-(1-ethoxyethoxy)ethyl-1-ethoxy-1-(2-phenylethoxy)ethane, cyclohexyl phenyl ethyl ether, 1-(4-isopropylcyclohexyl)ethanol, bicyclo[2.2.1]heptane-2-ethyl-5-methoxytricyclo[2.2.1.0.2.6]heptane, and bicyclo[2.2.1]heptane-2-ethyl-6-methoxytricyclo[2.2.1.0.2.6]heptane. Essential oils (and ingredients thereof) of plants used in perfumes and fragrances, such as spearmint oil, peppermint oil, lemon oil, orange oil, sage oil, rosemary oil, cinnamon oil, pimento oil, cinnamon leaf oil, perilla oil, wintergreen oil, clove oil, and eucalyptus oil, may also be used.

Any suitable concentration of perfume agent may be used in a perfume formulation. In certain implementations, a perfume agent may be present in a concentration from about 0.01% to about 10%, for example from about 0.02% to about 1%, or for example from about 0.05% to about 0.5%. One or more perfume agents may be used in a given perfume formulation.

A stable formulation may also comprise an N-halogenated or N,N-dihalogenated amine compound, a water-swellable polymer, and a perfume agent. By way of example and not limitation, a stable formulation may comprise 1% N,N-dichloro-2,2-dimethyltaurine in 1% AA-1 and 0.1% cineole. In another example, a formulation may comprise 0.3% N,N-dichloro-2,2-dimethyltaurine in 1% Polyox® 205 and 0.2% 3-octanone. Other examples are given in Examples 2-5 below.

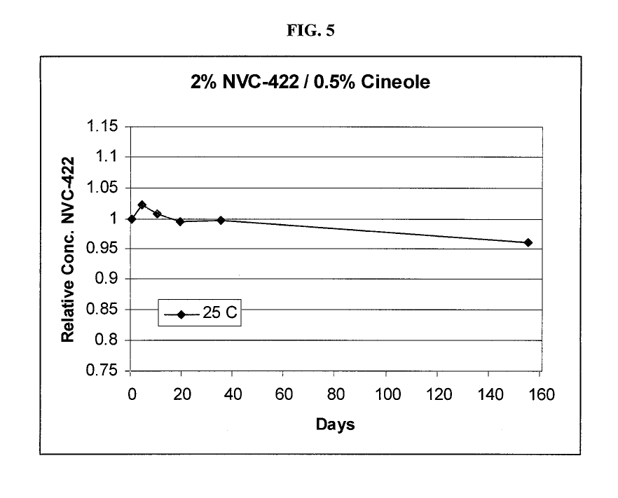

Referring to FIG. 5, an aqueous formulation of 2% NVC-422 in 0.5% cineole has 95% stability for at least 155 days at about 25° C. Formulations using concentrations of 0.1%, 0.2%, 0.3% and 0.4% cineole also met this stability profile. Aqueous formulations of 2% NVC-422 in 0.1%, 0.2%, 0.3%, 0.4%, and 0.5% 3-octanone each had 90% stabilities for at least 132 days at about 25° C.

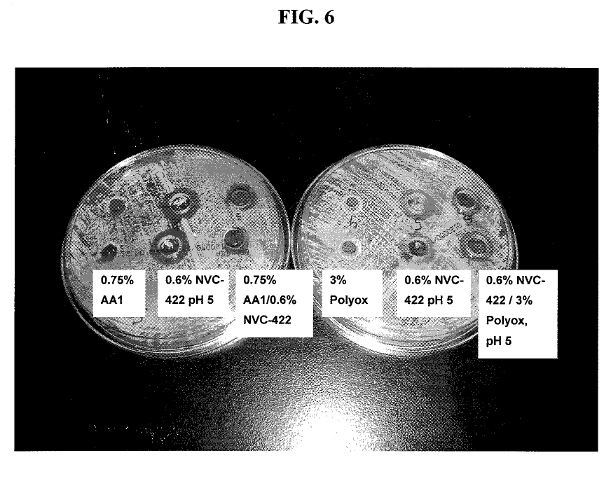

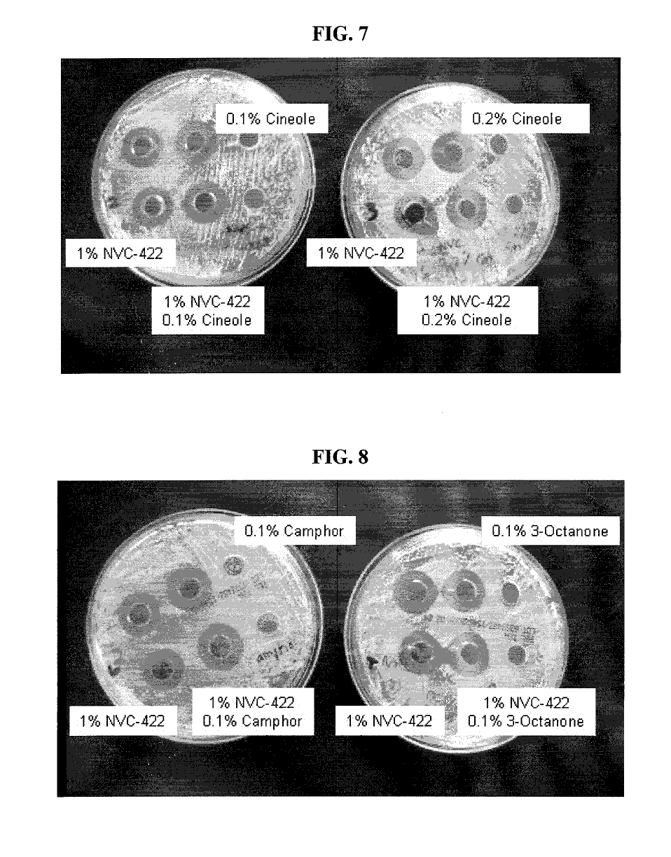

The antimicrobial activity of formulations described herein may be shown by radial diffusion assays. FIG. 6 shows the results of a radial diffusion assay of 0.6% NVC-422 alone and in formulations of 0.75% AA-1 (plate at left) and 3% Polyox® 205 (plate at right); the activity of the polymer alone is shown for comparison (at the left of each plate). FIGS. 7-8 show similar results of several perfume formulations of NVC-422. See Examples 7-8 for further detail.

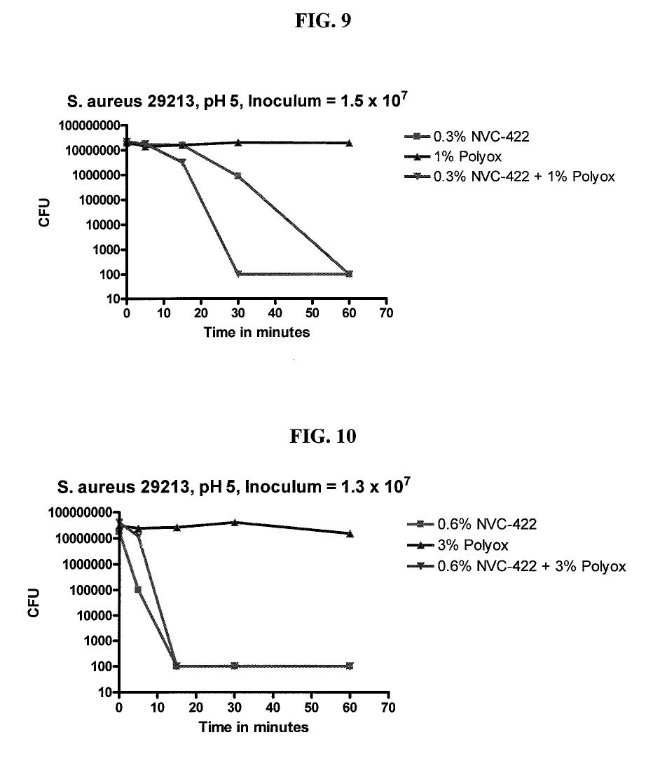

Certain polymer formulations of an N-halogenated or N,N-dihalogenated amine compound may have enhanced antimicrobial (e.g. antibacterial, antiviral, antifungal, etc.) activity as compared to solutions containing the an N-halogenated or N,N-dihalogenated amine compound alone. For example, FIG. 9 shows that a formulation of 0.3% NVC-422 in a 1% Polyox® WSR-205 (“Polyox 205”, or “Polyox” in certain figures) kills S. aureous more rapidly than when 0.3% NVC-422 is used alone. However, not all ratios of N-halogenated or N,N-dihalogenated amine compound to polymer show such enhanced activity. FIG. 10 shows that a formulation of 0.6% NVC-422 in 3% Polyox® 205 kills S. aureous at about the same rate as a solution of 0.6% NVC-422 with no polymer.

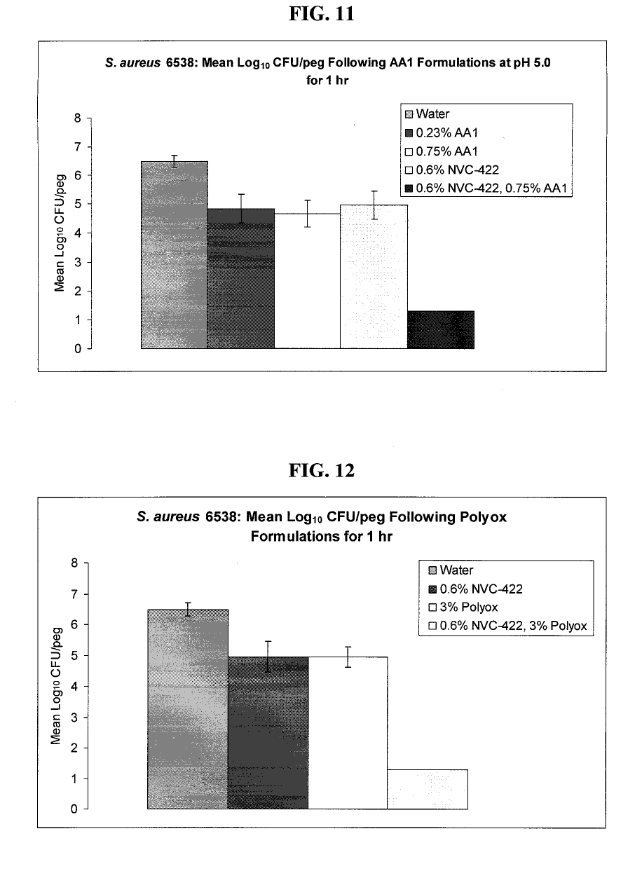

Referring to FIG. 11, a greater amount of S. aureous was killed when exposed to a formulation of 0.6% NVC-422 in 0.75% AA-1 than when exposed to either 0.6% NVC-422 (in aqueous solution) or a 0.75% solution of AA-1 alone. See also Example 9. FIG. 12 illustrates enhanced activity of a formulation of 0.6% NVC-422 in 3% Polyox® 205. It can clearly be seen that the degree of killing of S. aureous in these enhanced formulation examples is greater than a simple additive effect of the individual constituents of the formulations.

Formulations described herein may be used as antimicrobial formulations for application to a subject, and be useful in a method of preventing or treating an infection caused by a bacterial, a microbial, a sporal, a fungal or a viral activity, the method comprising the administration of an effective amount of the formulation. Such formulations may offer improved adhesion, activity, sustained release of the antimicrobial agent, and other properties in comparison to application of the antimicrobial agent with no such polymer. These formulations may be applied to external areas of a subject, such as the skin, hair, and nails, to mucous membranes such as the buccal mucosa, esophageal mucosa, gastric mucosa, intestinal mucosa, olfactory mucosa, oral mucosa, bronchial mucosa, uterine mucosa, and other areas of the body including the eye, urethra, rectum and vagina. For example, a formulation of 1.5% NVC-422 in 1% AA-1 may be used in a method to treat a bacterial skin infection (e.g. acne, cellulitis, eethyma, folliculitus, furunculosis, and impetigo), the method comprising administering an effective amount of the formulation to the area of interest.

The formulations described herein may also be used as or part of a personal care or cosmetic article, such as a hand sanitizer, an antimicrobial wash or wipe, an antimicrobial antiperspirant or deodorant, a topical skin or wound disinfectant, a facial wash, a body wash, an acne treatment or anti-acne rinse, a feminine hygiene product, a shampoo, and a dental rinse (e.g. mouthwash).

The formulations described herein may also be useful in other applications, including controlling or reducing microbial growth in a solution or on a surface, e.g. as a contact lens cleanser, in bacterial inactivation, ophthalmic applications, general surgical preparation including oncology, surgical instrument disinfection, medical device and instrument disinfection, dental instruments disinfection and application in food sanitation including disinfection of surface areas.

The formulations described herein may also be useful for the treatment of fungal infections, such as acute or chronic Rhinosinusitis or other fungal infections such as Otitis, Dermatitis, Bronchititis, Pneumonia's such as Pneumocystis carinii, the fungal infections of sex organs, such as Colpitis, Endometritis, Balnitis, fungal infections of the gastrointestinal tract, such as Stomatitis, Oesophagitis, Enteritis, or fungal infections of the urethra, such as Pyelonephrititis, Ureteritis, Cystitis, or Urethritis. Furthermore, the formulations described herein may have antimicrobial activity against many other microorganisms, including Escherichia coli, Listeria monocytogenes, Staphylococcus aureus, methicillin-resistant S. aureus (MRSA), Pseudomonas aeruginosa, Lactobacillus, yeast, vancomycin-resistant enterococcus, molds, and spores, including spores of anthrax and cysts of Acanthamoeba. In particular, the formulations of the present invention may be useful in the treatment of several different strains of Bacillus anthracis. Vancomycin-resistant bacteria, MRSA, and others are easily destroyed by the compositions of the present invention. Examples of bacteria implicated in periodontal disease and destroyed by the compounds of this invention are Bacteriodes gingivalis, B. intermedius, Actinomyces actinomycetemcomitans and B. forsythus. Examples of bacteria that are implicated in mastitis (infection of cow udder) and killed by the compounds are Streptococcus agalactiae and Streptococcus infantarius. Other applications may be envisaged.

### EXAMPLES

### Example 1

### AA-1 Gel Formulation

A formulation of 1% N,N-dichloro-2,2-dimethyltaurine in 1% AA-1 was prepared as follows. A 1.5% (w/v) solution of Noveon® AA-1 Polycarbophil was prepared by slowly adding the gelling agent to water while stirring to prevent clumping of the gelling agent. A 4% solution of N,N-dichloro-2,2-dimethyltaurine (w/v) was prepared separately. Amounts of the two solutions were then mixed to form a 1% N,N-dichloro-2,2-dimethyltaurine/1% AA-1 solution. The pH of the solution was adjusted to about 5.0 using HCl (and NaOH if necessary).

### Example 2

### Cineole Formulation

A 1% solution of N,N-dichloro-2,2-dimethyltaurine (“NVC-422”) in 0.2% cineole was prepared by mixing 1% NVC-422 (w/v) in 100 ml of 0.9% saline (NaCl) solution, followed by the addition of 0.2% 1,8-cineole (v/v). The resulting solution was clear and colorless, and had a spicy, eucalyptus-like smell.

### Example 3

### 3-Octanone Formulation

A 1% solution of NVC-422 in 0.5% 3-octanone was prepared by mixing 1% NVC-422 (w/v) in 100 ml of 0.9% saline (NaCl) solution, followed by the addition of 0.5% 3-octanone (v/v). The resulting solution was clear and colorless, and had a strong, sweet, floral smell.

### Example 4

### Camphor Formulation

A 1% solution of NVC-422 in 0.1% camphor was prepared by mixing 1% NVC-422 (w/v) in 100 ml of 0.9% saline (NaCl) solution, followed by the addition of 0.1% camphor (w/v). The resulting solution was clear and colorless, and had a woody, vanilla or eucalyptus-like smell.

### Example 5

### Gel for Dermatological Application

A gel for hand sanitization, acne rinse, or other dermatological application was prepared as follows. A 1% NVC-422/1% AA-1 solution was prepared according to Example 1. About 0.1% cineole (v/v) was then added, and the resulting formulation was mixed thoroughly.

### Example 6

### Time-Kill Assays of Antimicrobial Gel Formulations

Isolated S. aureus colonies were picked into 5 mL Tryptic Soy Broth (TSB) and grown for 4-6 hours at 37° C. Stock titers were determined by drop plate method. A working stock of S. aureus was prepared by dilution of the culture to a final inoculum of 1.5×10⁸ colony forming units (CFU) per mL in sterile pH 4.0 saline. 900 μL of each tested formulation was placed in sterile glass tubes. A 0.1 mL aliquot of S. aureus suspension containing 1.5×10⁸ CFU/mL was inoculated into the test sample container to give a final inoculum of 1.5×10⁷ CFU/mL. The inoculated formulation was immediately mixed for 3 seconds following inoculation using a vortex and incubated at room temperature. A plate count was performed on the inoculated samples after 0, 5, 15, 30 and 60 minutes following inoculation as follows:
0.1 mL of the inoculated antimicrobial test agent was placed into 0.9 mL of the D/E neutralizing broth
A 0.1 mL aliquot of each sample in D/E broth was drop plated on appropriately labeled agar dishes

All plates were incubated at 37° C. for 24 hours for bacterial growth assessment. At the conclusion of the incubation period the number of colonies present on the plates was counted. The total number of survivors at each time point was calculated by multiplying the CFU count obtained by the dilution factor.

The results shown in graphical form in FIG. 3 show that treatment with 0.3% NVC-422/1% Polyox® resulted in complete kill in 30 min, while treatment with 0.3% NVC-422 alone resulted in complete kill in 1 hr. 1% Polyox® alone (placebo) did not show any antibacterial activity.

The results shown in graphical form in FIG. 4 show that treatment with 0.6% NVC-422/3% Polyox® and with 0.6% NVC-422 alone resulted in complete kill in 15 min. 1% Polyox® alone (placebo) did not show any antibacterial activity.

### Example 7

### Radial Diffusion Assay of Antimicrobial Gel Formulations

Vials containing 0.6% NVC-422 (Drug Control), liquid, pH=5.0; 3% Polyox® WSR 205 (Placebo) Liquid, R.T. pH=5.0; 0.6% NVC-422/3% Polyox® WSR 205, Liquid, R.T., pH=5.0; 0.75% AA-1 (Placebo), 0.6% NVC-422/0.75% AA-1 and 0.23% AA-1 (alternate placebo) were prepared. The first three test articles appeared as clear liquids, while the last three appeared as gels. The samples were tested in the radial diffusion assay against S. aureus 29213. 100 μl of each formulation were tested in duplicate on two plates each (n=4 total).

Results are shown on FIG. 6. These results may be summarized as follows:
0.6% NVC-422, pH 5 produced a clearing zone of 15.3 mm (Table 1).
3% Polyox® and 0.75% AA-1 without NVC-422 (placebos) did not produce any clearing zones.
Test formulations (0.6% NVC-422/3% Polyox® and 0.6% NVC-422/0.75% AA-1) produced clearing zones of 12.5 mm each, indicating that activity of NVC-422 in formulations may be somewhat reduced compared to that of NVC-422 in saline solution.

TABLE 1
Clearing zone diameters, mm

| Test Article | Sample 1 | Sample 2 | Sample 3 | Sample 4 | Average +/− SD |
|---|---|---|---|---|---|
| 0.75% AA-1 | — | — | — | — | — |
| 0.6% NVC-422, | 15 | 16 | 16 | 14 | 15.3 +/− 1.0 |
| pH 5 | | | | | |
| 0.75% AA-1 + | 14 | 14 | 12 | 10 | 12.5 +/− 1.9 |
| 0.6% NVC-422 | | | | | |
| 3% Polyox ® | — | — | — | — | — |
| 3% Polyox ® + | 13 | 12 | 12 | 13 | 12.5 +/− 0.6 |
| 0.6% NVC-422 | | | | | |

### Example 8

### Radial Diffusion Assay of Antimicrobial Perfume Formulations

Radial diffusion assays showing the antimicrobial activity of N,N-dichloro-2,2-dimethyltaurine (“NVC-422”) were performed as follows. The formulation samples were tested in the radial diffusion assay against S. aureus ATCC 6538. A standard culture of S. aureus was grown in tryptic soy broth (TSB) for 4-6 hrs. The culture was adjusted to 1×10⁸ CFU/mL by absorbance reading at 600 nm. Each tryptic soy agar (TSA) plate was inoculated using a sterile cotton swab and was allowed to dry for 2 hrs in an incubator at 35° C. A total of 6 wells per plate (using 8 mm biopsy punches) were made and 100 μl of formulation was pipetted into each well. The TSA plates were incubated overnight at 35° C. The average diameter of clearing zones was measured the next day. Each formulation was applied to two wells. Referring to FIGS. 7-8, the left rows show the activity of NVC-422 alone, the middle rows show the activity of NVC-422 in a perfume formulation, and the right rows show activity of the perfume agent alone (negative control). These results show that NVC-422 has antibacterial activity in the perfume formulations tested.

### Example 9

### Calgary Biofilm Device (MBEC™) Assay

Methods:

Viable Cell Count

S. aureus 6538 biofilm was grown in “minimum biofilm eliminating concentration” (“MBEC”) plates for 24 hr at 35° C. in TSB prior to treatment using the following method:
Prepare a standard inoculum by transferring a well isolated colony from a stock agar plate to 4 to 5 ml of suitable broth medium specified in the ATCC product specification sheet for each individual organism. Incubate at 37° C. on a shaker for 4-6 hr.
Add ˜2 ml of standard inoculum into a 50 ml conical tube containing 20 ml broth medium. Adjust inoculum concentration to reach an absorbance reading of 0.04-0.05 at 600 nm. Shake tube gently and transfer into sterile solution basin. Pipet 150 μl diluted inoculum into each well of the Calgary Biofilm device plate. Place plate on a shaker and incubate plate at 37° C. The shaker should be set to between 100 and 150 revolutions per minute (RPM).
Determine the actual bacterial titer on the diluted standard inoculum by agar plating of serial dilution, or other method such as comparison with turbidity standards. Diluted inoculum should be about 10⁶ CFU/ml.
Pipette 200 μL PBS, 200 μL D/E Neutralizing broth, 270 μl D/E Neutralizing broth into each wells of black Costar plates.
After 24 hr, remove lid and rinse in a 200 μl, PBS plate for 1 minute to rinse off planktonic cells. Place the lid in another plate containing 200 μL D/E Neutralizing broth and sonicate in water bath for 15 min. After sonication, remove 30 μL from the dispersed biofilm and serially dilute in a plate containing 270 μl D/E Neutralizing broth.

The MBEC lid containing pegs coated with biofilm was first rinsed in a plate containing 200 μl of PBS in each well for 1 minute, prior to treatment with a plate containing formulations for 60 minutes total. The lid was neutralized in a plate containing 200 μl D/E Neutralization broth in each well and was immediately sonicated for 15 minutes. Serial dilution was performed to calculate viable cell counts.

Absorbance Reading

S. aureus 6538 biofilm was grown in MBEC plates for 24 hr at 35° C. for 24 hr in TSB prior to treatment using the method describe above. The MBEC lid containing pegs coated with biofilm was first rinsed in a plate containing 2000 of PBS in each well for 1 minute, prior to treatment with a plate containing formulations for 60 minutes total. The lid was neutralized in a plate containing 200 μl D/E Neutralization broth in each well for 1 minute before transferring the lid into a plate containing 150 μl TSB in each well. Plate was incubated overnight at 35° C. Absorbance was read at 650 nm the following day.

Results:

Vials containing 0.6% NVC-422 (PH0811/37-1) 0.23% AA-1 (PH0811/36-2) 0.75% AA-1 (PH0811/36-3), 0.6% NVC-422/0.75% AA-1 (PH0811/36-4), 3% Polyox® WSR 205 (PH0811/36-5), 0.6% NVC-422/3% Polyox® WSR 205 (PH0811/36-6) were prepared. All solutions were at pH 5.0. All samples were simultaneously tested in the MBEC 96-well assay against S. aureus 6538.

Viable cell count results (an average of two independent experiments, n_{wells}≦8) are shown in FIG. 8 and FIG. 9. Referring to FIG. 8, treatment with a formulation of 0.6% NVC-422 in 0.75% AA-1 resulted in an average of about 5 log₁₀ reduction compared to water. Treatments with 0.6% NVC-422 alone, and 0.23% or 0.75% AA-1 alone resulted in approximately 1.5 log₁₀ reductions compared to water. Referring to FIG. 9, treatment with a formulation of 0.6% NVC-422 in 3% Polyox® resulted in an average of about 5 log₁₀ reduction when compared to water. Treatments with 0.6% NVC-422 alone and 3% Polyox® alone resulted in about 1.5 log₁₀ reductions compared to water.

Table 2 shows absorbance readings at 650 nm. Absorbance values of greater than 0.1 indicated incomplete biofilm eradication.

TABLE 2
A₆₅₀ readings of 0.6% NVC-422 in 0.3% Polyox ® and
0.75% AA-1 formulation testings in MBEC assay

| | Treatment | | | | | | |
|---|---|---|---|---|---|---|---|
| | 0.6% | 0.23% | 0.75% | 0.6% NVC-422 + | 3% | 0.6% NVC-422 + | |
| | NVC-422 | AA-1 | AA-1 | 0.75% AA-1 | Polyox | 3% Polyox | Water |
| Ave. Abs | 0.48 | 0.66 | 0.48 | 0.08 | 0.50 | 0.05 | 0.67 |
| at 650 nm | | | | | | | |
| StDev | 0.34 | 0.08 | 0.17 | 0.01 | 0.09 | 0.00 | 0.02 |
| CV | 0.71 | 0.12 | 0.35 | 0.12 | 0.18 | 0.04 | 0.03 |

In summary, treatment with 0.6% NVC-422/3% Polyox or 0.6% NVC-422/0.75% AA-1 resulted in 4 log₁₀ reduction. Treatment with 0.6% NVC-422 alone resulted in 1 log₁₀ reduction when compared to water. Treatment with 3% Polyox®, 0.23% AA-1 or 0.75% AA-1 alone had either slight antibacterial activity or mechanical removal of biomass from pegs due to viscosity of gels. Absorbance readings were consistent with viable cell count result whereby there was complete biofilm eradication with 0.6% NVC-422/3% Polyox® or 0.6% NVC-422/0.75% AA-1.

A number of implementations of the invention have been described. Nevertheless, it will be understood that various modifications may be made without departing from the spirit and scope of the invention. Accordingly, other implementations are within the scope of the following claims.

What is claimed is:

1. A formulation comprising:
a compound of Formula (I)
A-C(R¹R²)R(CH₂)ₙC(R³R⁴)—Y—Z (I)
or a derivative thereof, wherein
A is hydrogen, HalNH— or Hal₂N—, wherein Hal is a halogen selected from the group consisting of chloro, bromo and iodo;
R¹ is hydrogen or an optionally substituted group selected from the group consisting of alkyl, cycloalkyl, heteroalkyl, haloalkyl, aryl, heteroaryl and heterocycloalkyl and —COOH;
R² is hydrogen or an optionally substituted group selected from the group consisting of alkyl, cycloalkyl, heteroalkyl, haloalkyl, aryl, heteroaryl and heterocycloalkyl, or R′ and R² together with the carbon atom to which they attach form an optionally substituted cycloalkyl or heterocycloalkyl group;
R is a carbon-carbon single bond or a divalent cycloalkylene radical with three to six carbon atoms,
n is 0 or an integer from 1 to 13;
R³ and R⁴ are each independently selected from the group consisting of hydrogen, fluoro, —NH₂, —NHHal, NHal₂, and an optionally substituted group selected from the group consisting of alkyl, cycloalkyl, heteroalkyl, aryl, heteroaryl and heterocycloalkyl groups;
Y is selected from a group consisting of a single bond, —O—, —CF₂—, —CHF—, —C(═O)—, —C(═O)O—, —OC(═O)—, —C(═O)NR^{a}—, —NR^{a}C(═O)—, P(═O)(OR^{b})O—, —OP(═O)(OR^{b})—, —P(═O)(OR^{b})NR^{c}—, —NR^{c}P(═O)(OR^{b})—, —S(—O)₂, —S(═O)₂O—, —OS(═O)₂—, —S(═O)₂NR^{d}—, —NR^{d}S(═O)₂—, or heteroarylene wherein R^{a}, R^{b}, R^{c} and R^{d} are each independently selected from the group consisting of hydrogen, and optionally substituted alkyl, aryl, cycloalkyl, heteroalkyl, heteroaryl and heterocycloalkyl; a divalent (C₁₋₁₈)alkylene group in which, optionally, one or two methylene groups are replaced with a mono- or di-substituted methylene group; and a divalent (C₁₋₁₈)heteroalkylene group wherein the divalent (C₁₋₁₈)heteroalkylene group is a divalent (C₁₋₁₈)alkylene group in which, optionally, one or two methylene groups are replaced with 1 or 2 —NR′—, —O—, —S—, —S(═O)—, >C═O, —C(═O)O—, —OC(═O)—, —C(═O)NH—, —NHC(═O)—, —C(═O)NR′—, —NR′C(═O)—, —S(═O)₂—, —S(═O)₂NR′—, —S(═O)₂NH—, —NR′S(═O)₂— or —NHS(═O)₂— group, wherein R′ is selected from the group consisting of hydrogen, Cl, Br, and optionally substituted alkyl, aryl, cycloalkyl, heteroalkyl, heteroaryl, heterocycloalkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)arylC(═O)— and (C₆₋₁₀)aryl(C₁₋₄alkylC(═O)— wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀aryl(C₁₋₄)alkyl, heteroaryl comprising 4 to 10 ring atoms with at least one heteroatom selected from O, S and N in the ring, or heterocycloalkyl(C₁₋₄)alkyl, the heterocycloalkyl group containing 2-10 carbon atoms and 1 to 4 heteroatoms selected from N, O or S;
Z is selected from the group consisting of hydrogen, —CO₂H, —CONH₂, —SO₃H, —SO₂NH₂, —P(═O)(OH)₂, —B(OH)₂, —[X(R⁵)(R⁶)R⁷]Q, —S(═O)₂NR^{c}R^{d}, —S(═O)₂NHC(═O)R^{e}, S(═O)₂C(═O)NR^{c}R^{d}, —S(═O)₂NR^{c}C(═O)NR^{c}R^{d} and —S(═O)₂(N═)C(OH)NR^{c}R^{d} wherein R^{c} and R^{d} are each independently hydrogen or is independently selected from the group consisting of (C₁₋₅)alkyl, (C₃₋₆)cycloalkyl, (C₁₋₅)alkylNHC(═O)—, (C₆₋₁₀)arylC(═O)—, (C₆₋₁₀)aryl(C₁₋₄)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S, and R^{e} is hydrogen or is selected from the group consisting of (C₁₋₅)alkyl, (C₃₋₆)cycloalkyl, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S; or a salt, an amine oxide thereof, or a derivative or a bioisostere or a prodrug thereof;
X is selected from the group consisting of N, P, and S;
Q is a counterion or is absent;
R⁵ and R⁶ are each independently selected from the group consisting of alkyl, aryl, cycloalkyl, heteroalkyl, heteroaryl and heterocycloalkyl, each of which may be optionally substituted; or R⁵ and R⁶ together with the X atom to which they are attached form heterocycloalkyl group, which may be optionally substituted; and
R⁷ is alkyl, aryl, cycloalkyl, heteroalkyl, heteroaryl or heterocycloalkyl, each of which may be optionally substituted, and may further be 0 when X is N, with the proviso that R⁷ is absent when X is S;
with the proviso that if R is a divalent cycloalkylene radical, n will not exceed the integer 11;
wherein the compound is dispersed in a water-swellable polymer;
and wherein the compound is 90% stable for at least 30 days at about 25° C.

2. The formulation of claim 1, wherein the compound of formula (I) is a compound of Formula (IA)
A-C(R¹R⁰)R(CH₂)ₙ—C(R³R⁴)—X′ (IA)
or a derivative thereof, wherein
A is hydrogen, HalNH— or Hal₂N— wherein Hal is halogen selected from the group consisting of chloro, bromo and iodo;
R¹ is hydrogen, C₁₋₆alkyl or the group —COOH;
R⁰ is hydrogen or C₁₋₆alkyl; or R¹ and R⁰ together with the carbon atom to which they attach form a (C₃-C₆)cycloalkyl ring;
R is a carbon-carbon single bond or a divalent cycloalkylene radical with three to six carbon atoms;
n is 0 or an integer from 1 to 13;
R³ is hydrogen, C₁₋₆alkyl, —NH₂ or —NHal₂;
R⁴ is hydrogen or C₁₋₆alkyl; and
X′ is hydrogen, —COOH, —CONH₂, —SO₃H, —SO₂NH₂, —P(═O)(OH)₂ or —B(OH)₂;
with the proviso that if R is a divalent cycloalkylene radical n will not exceed the integer

3. The formulation of claim 1 wherein:
A is HalNH— or Hal₂N—;
R¹ and R² are each independently optionally substituted alkyl;
R is a carbon-carbon single bond;
n is an integer from 1 to 3;
R³ and R⁴ are both hydrogen;
Y is a single bond;
Z is —SO₃H or —[X(R⁵)(R⁶)R⁷]Q, wherein X is N, S or P; R⁵, R⁶, and R⁷ are independently optionally substituted alkyl; and Q is a counterion or absent.

4. The formulation of claim 1 wherein the compound of Formula (I) is selected from the group consisting of:
N,N-dichlorotaurine;
N,N-dichloro-2-methyltaurine;
N,N-dichloro-2,2,3,3-tetramethyl-β-alanine;
N,N-dichloro-2,2-dimethyltaurine;
N,N-dichloro-1,1,2,2-tetramethyltaurine;
N,N-dibromo-2,2-dimethyltaurine;
N,N-dibromo-1,1,2,2-tetramethyltaurine;
N,N-diiodotaurine;
N,N-dichloro-3,3-dimethylhomotaurine;
N,N-dichloro-2-methyl-2-amino-ethanesulfonic acid;
N,N-dichloro-1-methyl-ethanesulfonic acid;
N,N-dichloro amino-trimethylene phosphonic acid;
N,N-dibromo-2-amino-5-phosphonopantanoic acid;
N,N-dichloro amino-ethylphosphonic acid dimethylester;
N,N-dichloro amino-ethylphosphonic acid diethylester;
N,N-dichloro-1-amino-1-methylethane phosphonic acid;
N,N-dichloro-1-amino-2-methylethane phosphonic acid;
N,N-dichloro-1-amino-2-methylpropane phosphonic acid;
N,N-dichloro-leucine phosphonic acid;
N,N-dichloro-4-amino-4-phosphonobutyric acid;
(±)N,N-dichloro-2-amino-5-phosphonovaleric acid;
N,N-dichloro-(+)-2-amino-5-phosphonovaleric acid;
N,N-dichloro d,l-2-amino-3-phosphonopropionic acid;
N,N-dichloro-2-amino-8-phosphonooctanoic acid;
N,N-dichloro-leucine boronic acid;
N,N-dichloro-β-alanine boronic acid;
N-chlorotaurine;
N-chloro-2-methyltaurine;
N-chloro-2,2,3,3-tetramethyl-β-alanine;
N-chloro-2,2-dimethyltaurine;
N-chloro-1,1,2,2-tetramethyltaurine;
N-bromo-2,2-dimethyltaurine;
N-bromo-1,1,2,2-tetramethyltaurine;
N-iodotaurine;
N-chloro-3,3-dimethylhomotaurine;
N-chloro-2-methyl-2-amino-ethanesulfonic acid; and
N-chloro-1-methyl-ethanesulfonic acid,
N-chloro amino-trimethylene phosphonic acid;
N-bromo-2-amino-5-phosphonopantanoic acid;
N-chloro amino-ethylphosphonic acid dimethylester;
N-chloro amino-ethylphosphonic acid diethylester;
N-chloro-1-amino-1-methylethane phosphonic acid;
N-chloro-1-amino-2-methylethane phosphonic acid;
N-chloro-1-amino-2-methylpropane phosphonic acid;
N-chloro-leucine phosphonic acid;
N-chloro-4-amino-4-phosphonobutyric acid;
(±)N-chloro-2-amino-5-phosphonovaleric acid;
N-chloro-(+)2-amino-5-phosphonovaleric acid;
N-chloro d,l-2-amino-3-phosphonopropionic acid;
N-chloro-2-amino-8-phosphonooctanoic acid;
N-chloro-leucine boronic acid;
N-chloro-β-alanine boronic acid;
(1-(dichloroamino)cyclohexyl)methanesulfonic acid;
(1-(chloroamino)cyclohexyl)methanesulfonic acid;
2-(chloroamino)-N,N,N-2-tetramethylpropan-1-ammonium chloride;
2-(dichloroamino)-N,N,N-2-tetramethylpropan-1-ammonium chloride;
3-(chloroamino)-N,N,N-3-tetramethylbutan-1-ammonium chloride;
3-(dichloroamino)-N,N,N-3-tetramethylbutan-1-ammonium chloride;
1-(2-(dichloroamino)-2-methylpropyl)-1-methylpiperidinium chloride;
1-(2-(chloroamino)-2-methylpropyl)-1-methylpiperidinium chloride;
(2-(dichloroamino)-2-methylpropyl)dimethylsulfonium chloride;
(2-(chloroamino)-2-methylpropyl)dimethylsulfonium chloride;
(4-(dichloroamino)-4-methylpentyl)trimethylphosphonium chloride;
(4-(chloroamino)-4-methylpentyl)trimethylphosphonium chloride;
3-(3-(dichloroamino)-3-methylbutylsulfonyl)-N,N,N-trimethylpropan-1-aminium chloride;
3-(3-(chloroamino)-3-methylbutylsulfonyl)-N,N,N-trimethylpropan-1-aminium chloride;
2-(3-(dichloroamino)-3-methylbutylsulfonyl)-N,N,N-trimethylethanaminium chloride; and
2-(3-(chloroamino)-3-methylbutylsulfonyl)-N,N,N-trimethylethanaminium chloride.

5. The formulation of claim 1 wherein the water-swellable polymer comprises poly(ethylene oxide), poly(acrylic acid), or a combination thereof.

6. The formulation of claim 1 wherein the concentration of the compound is from about 0.01% to about 5.0% by weight, wherein the concentration of the polymer is from about 0.01% to about 10.0% by weight, and having a pH from about 3.0 to about 9.0.

7. The formulation of claim 1, in the form of a cream, gel, lotion, ointment, paste or aerosol.

8. A formulation, comprising
a compound of Formula (I) of claim 1; and
a perfume agent,
wherein the compound is 90% stable for at least 30 days at about 25° C.

9. The formulation of claim 8 wherein the perfume agent is selected from the group consisting of menthol, anethole, carvone, eugenol, limonene, ocimene, n-decylalcohol, citronellol, a-terpineol, methyl salicylate, methyl acetate, citronellyl acetate, cineole (e.g. 1,8-cineol, also known as eucalyptol), camphor, linalool, ethyl linalool, vanillin, thymol, isoamyl phenyl ether, isoborneol, isoborneol methyl ether, 2,2-dimethylbicyclo[2.2.1]heptane-3-carboxylic acid methyl ester, 2-tertiary pentyl cyclohexanyl acetate, 7-octen-2-ol-2,6-dimethyl acetate, 1-methyl-4-isopropyl cyclohexan-8-yl acetate, tetrahydrogeraniol, 2,6-dimethylheptan-2-ol, diphenyl methane, diphenyl oxide, alpha-fenchyl acetate, 1,3-dioxane-2,4,6-trimethyl-4-phenyl, 4-methyl-2-(2-methylpropyl)tetrahydro-2H-pyran-4-ol, ethyl tricyclo[5.2.1.02,6]decan-2-carboxylate, 2-methyldecanonitrile, 2-butyl-4,4,6-trimethyl-1,3-dioxane, 2-butyl-4,4,6-trimethyl-1,3-dioxane, limetol, 3,12-tridecadiene nitrile, methyl lavender ketone, octanal dimethyl acetal, orange flower ether (i.e. 4-(1-methoxy-1-methylethyl)-1-methylcyclohexene), p-tertiary butyl cyclohexanol, benzene pentanol, gamma-bethyl, 3-octanol, 3,7-dimethyl-3-octanol, 2,6-dimethyl-2-octanol, 2-octanone, 3-octanone, thymyl methyl ether, ortho-tertiary butyl cyclohexanyl acetate, benzene, [2-(1-ethoxyethoxy)ethyl-1-ethoxy-1-(2-phenylethoxy)ethane, cyclohexyl phenyl ethyl ether, 1-(4-isopropylcyclohexyl)ethanol, bicyclo[2.2.1]heptane-2-ethyl-5-methoxytricyclo[2.2.1.0.2.6]heptane, bicyclo[2.2.1]heptane-2-ethyl-6-methoxytricyclo[2.2.1.0.2.6]heptane, spearmint oil, peppermint oil, lemon oil, orange oil, sage oil, rosemary oil, cinnamon oil, pimento oil, cinnamon leaf oil, perilla oil, wintergreen oil, clove oil and eucalyptus oil.

10. The formulation of claim 1, further comprising a perfume agent selected from the group consisting of cineole and 3-octanone.

11. The formulation of claim 1, wherein:
the compound of formula (I) is selected from the group consisting of N,N-dichloro-2,2-dimethyltaurine, N-chloro-2,2-dimethyltaurine; 2-(3-(dichloroamino)-3-methylbutylsulfonyl)ethanesulfonic acid; 2-(4-(dichloroamino)-4-methylpentylsulfonyl)ethanesulfonic acid; 3-(dichloroamino)-N,N,N,3-tetramethylbutan-1-aminium chloride; 1-(2-(dichloroamino)-2-methylpropyl)-1-methylpiperidinium chloride; 3-(dichloroamino)-N,N-diethyl-N,3-dimethylbutan-1-aminium chloride; and 3-(dichloroamino)-N,N,N-triethyl-3-methylbutan-1-aminium chloride; and
the polymer is poly(ethylene oxide), poly(acrylic acid), or a combination thereof.

12. The formulation of claim 11 further comprising a perfume agent selected from the group consisting of cineole and 3-octanone.

13. A personal care or cosmetic article selected from the group comprising a hand sanitizer, antimicrobial wash or wipe, topical skin or wound disinfectant, facial wash, body wash, an acne treatment or anti-acne rinse, a feminine hygiene product, a shampoo, and a dental rinse, the article comprising a formulation of claim 1.

14. A method of treating an infection caused by a bacterial, a microbial, a sporal, a fungal or a viral activity, the method comprising the administration of a formulation of claim 1.

15. The method of claim 14 wherein the infection is a bacterial skin infection.
